SEQUENCE LISTING (3 sequences)

SEQ ID NO: 1 - DNA, 1389 bp, synthetic construct
  misc_feature 1..1389: note = FKBP12-SB100X
  source 1..1389: mol_type = other DNA
atgggcgtgcaggtcgagacaatctctcctggcgacggcagaacattccccaagaggggccagacatgcgtggtgcactataccggcatgctggaagatggcaagaaggtggacagcagccgggacagaaacaagcccttcaagttcatgctgggcaagcaagaagtgatcagaggctgggaagagggcgtcgcccagatgtctgttggacagagagccaagctgacaatcagccccgattacgcctatggcgccacaggacaccctggcatcattcctccacatgccacactggtgttcgacgtggaactgctgaagcccgagaaacttggaggcggagcaccagctgttggcggcggaccaaagatgggcaagagcaaagagatcagccaggacctgcggaagcggatcgtggatctgcacaagtctggctctagcctgggcgccatcagcaagagacttgcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccactcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatggacgcggtgcagtatgtggatatattgaagcaacatctcaagacatcagtcaggaagttaaagcttggtcgcaaatgggtcttccaacacgacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaaattattgtgggaagcttgtggaaggctacccgaaacgtttgacccaagttaaacaatttaaaggcaatgctaccaaatactag

SEQ ID NO: 2 - DNA, 1539 bp, synthetic construct
  misc_feature 1..1539: note = ecDHFR-SB100X
  source 1..1539: mol_type = other DNA
atgatcagcctgatcgccgctctggccgtggattacgtgatcggcatggaaaacgccatgccttggaacctgcctgccgatctggcctggttcaagcggaacaccctgaacaagcccgtgatcatgggcagacacacctgggagtctatcggcagacctctgcctggccggaagaacatcatcctgagcagccagcctagcaccgacgacagagtgacatgggtcaagagcgtggacgaagccattgccgcttgcggagatgtgcctgagatcatggttatcggcggaggcagagtgatcgagcagttcctgcctaaggctcagaagctgtacctgacacacatcgacgccgaggtggaaggcgacacccactttccagactacgagcccgatgactgggagagcgtgttcagcgagttccacgatgccgacgctcagaacagccacagctactgcttcgagatcctggaaagaaagctcggaggcggagcccctgctgttggcggaggaccaaagatgggcaagagcaaagagatcagccaggacctgcggaagcggatcgtggatctgcacaagtctggctctagcctgggcgccatcagcaagagacttgcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccactcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatggacgcggtgcagtatgtggatatattgaagcaacatctcaagacatcagtcaggaagttaaagcttggtcgcaaatgggtcttccaacacgacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaaattattgtgggaagcttgtggaaggctacccgaaacgtttgacccaagttaaacaatttaaaggcaatgctaccaaatactag

SEQ ID NO: 3 - DNA, 1203 bp, synthetic construct
  misc_feature 1..1203: note = Degron-SB100X
  source 1..1203: mol_type = other DNA
atgcacaagaggagccacaccggcgagaggcccttccagtgcaaccagtgcggcgccagcttcacccagaagggcaacctgctgaggcacatcaagctgcacaccggcgagaagcccttcaagtgccacctgtgcaacaagctgggcggcggcgcccccgccgtgggcggcggccccaagatgggaaaatcaaaagaaatcagccaagacctcagaaaaagaattgtagacctccacaagtctggttcatccttgggagcaatttccaaacgcctggcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccactcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatggacgcggtgcagtatgtggatatattgaagcaacatctcaagacatcagtcaggaagttaaagcttggtcgcaaatgggtcttccaacacgacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaaattattgtgggaagcttgtggaaggctacccgaaacgtttgacccaagttaaacaatttaaaggcaatgctaccaaatactag

(19) United States
(12) Patent Application Publication
(10) Pub. No.: US 2025/0361284 A1
(43) Pub. Date: Nov. 27, 2025

(54) TRANSPOSASE FUSION PROTEINS FOR USE IN CELL AND GENE THERAPY

(71) Applicant: JULIUS-MAXIMILIANS-UNIVERSITÄT WÜRZBURG, Würzburg (DE)

(72) Inventors: Michael HUDECEK, Höchberg (DE); Narayanavari A. SUNEEL, Utrecht (NL)

(21) Appl. No.: 18/874,078

(22) PCT Filed: Jun. 13, 2023
(86) PCT No.: PCT/EP2023/065772

§ 371 Date: Dec. 11, 2024

(30) Foreign Application Priority Data
Jun. 13, 2022 (EP) 22178765.8

(51) Int. Cl. C07K 14/725; A61K 31/7105; A61K 35/12; A61K 40/11; C12N 5/10; C12N 9/06; C12N 15/62

(52) U.S. Cl. CPC C07K 14/7051; A61K 31/7105; A61K 35/12; A61K 40/11; C12N 5/10; C12N 9/003; C12N 15/62; C12Y 105/01003; C07K 2319/95; C12N 2510/00

(57) ABSTRACT

Fusion proteins and methods for conditionally fine-tuning transposase protein stability and activity for clinical applications are provided herein

### FIELD OF THE INVENTION

The invention generally relates to the development and use of novel transposase fusion proteins and control of their half-life and activity with pharmacologic agents. In particular, the invention relates to the use of these novel transposase fusion proteins for controlling transposon copy number and reducing genotoxicity in genetically engineered cells for adoptive immunotherapy, e.g., by introducing chimeric antigen receptors (CAR) into T cells for cancer immunotherapy in clinical applications.

### BACKGROUND OF THE INVENTION

Sleeping Beauty (SB) is a resurrected DNA transposon that is active in a wide range of vertebrates including humans (Ivics et al., 1997). The SB transposon system has two components, the transposon that carries the transposable DNA element between inverted terminal repeats (ITRs) and the transposase that catalyzes the excision and integration steps of transposition. The SB transposon system is a virus-free tool for gene transfer and genetic engineering and because of its ability to integrate therapeutic genes of interest into the genome of cells, it has been used in various applications in cell and gene therapy, including cellular immunotherapy in cancer, and gene therapy in neurologic and ocular disorders (Hudecek et al., 2017; Narayanavari et al., 2017).

Recently, the vectorization of SB transposase and transposon has been improved to support regulatory compliance and clinical use. For instance, vectorization of the SB transposon donor vector as minicircle DNA that lacks antibiotic resistance gene and origin of replication has been developed to enhance transposition rate (Monjezi et al., 2017), and SB transposase has been vectorized as mRNA instead of DNA, or has been supplied as recombinant protein in order to shorten the time during which host cells are exposed to the transposase (Monjezi et al., 2017; Querques et al., 2019; Wilber et al., 2006).

Virus-free gene delivery systems like the SB and PiggyBac (PB) transposon system are under investigation for making chimeric antigen receptor (CAR) T cells in cancer immunotherapy (Kebriaei et al., 2016; Magnani et al., 2020; Prommersberger et al., 2021; Ramanayake et al., 2015). Regulatory authorities like the European Medicines Agency (EMA) and the United States Food and Drug Administration (FDA) have set forth guidelines and proposed criteria that gene transfer systems ought to fulfil in order to support the clinical use of gene engineered cell therapies like CAR T cells (EMA/CAT/GTWP/671639/2008). Accordingly, in order to assess genomic safety and genotoxicity several parameters are typically considered, including (i) the genomic insertion profile, (ii) the vector copy number in the host cell genome and (iii) the presence of residual non-integrated gene transfer vector in the drug product (Hudecek et al., 2017; Prommersberger et al., 2021; Singh et al., 2014).

Regarding the genomic insertion profile—compared to other integrating vectors, the SB transposon system is thought to possess a relatively safe integration profile that can satisfy regulatory criteria (Gogol-Doring et al., 2016). Regarding the vector copy number—an approach to control and steer the transposon copy number in the host cell genome after SB transposition is desirable in order to reduce genotoxicity. In a study of SB-mediated gene transfer in human T cells, more than 20 transposon copies per T cell genome has been observed, which is considered too high (benchmark: 5 copies/genome) (Peng et al., 2009). Even with the use of short-lived mRNA to encode SB transposase, the transposon copy number in human T cells was between 8 to 12, which is still higher than desired (Miskey et al., 2019). Strategies to control the transposon copy number in host cells after SB transposition are currently lacking and are highly desired. Regarding the presence of SB transposase in the drug product—it was observed in a clinical trial wherein CAR T cell products were manufactured with plasmid-encoded SB transposon and transposase, that CAR T cells still contained SB transposase even after 21 days post-transfection and were administered to patients (Kebriaei et al., 2016). The presence of SB transposase in CAR T cells for such an extended period of time and that are administered to patients is not desired, because SB transposase protein is cytotoxic to cells and poses an excess risk of genotoxicity due to transposon remobilization (Galla et al., 2011; Riordan et al., 2014). Although SB has a relatively low frequency of remobilizing integrated transposons, the chance of remobilization should be curtailed (Riordan et al., 2014). Accordingly, there is a desire to control the activity and stability of SB transposase in order to control the ensuing transposon copy number and prevent transposon remobilization (Chan et al., 2017; de Macedo Abdo et al., 2020; Magnani et al., 2020; Torikai et al., 2017). The ability to control the amount and longevity of SB transposase protein that is available and active in host cells will enable and facilitate the use of SB transposition in pre-clinical and clinical applications of cell and gene therapy.

An appealing strategy to control transposon copy number and at the same time rapidly deplete SB transposase protein within host cells, is to reduce the half-life of the SB transposase protein by targeting it to the cellular protein degradation machinery. SB transposase protein is a highly stable protein with a half-life of approximately 72 to 80 hours under physiological conditions in cell culture (Geurts et al., 2003; Mates et al., 2009). Therefore, strategies to reduce the half-life of SB transposase are highly desired. Methods to conditionally regulate transcription of genes at the DNA level (for example the tetracycline regulated transactivator) are widely used but often suffer from leakiness and a temporal delay as previously transcribed mRNA continues to be translated into protein. Another approach is to use the RNA interference (RNAi) technology that destroys the target mRNA however, RNAi is only partially effective and also possess off-target effects. Overall, methods that function at the pre-translational level (i.e. at the DNA and mRNA level) are typically slow, only partially effective and cannot deplete already existing protein in the host cells. These problems could be overcome by targeting proteins directly. All eukaryotic systems are equipped with quality control systems that rapidly degrade misfolded proteins through the 26S proteasome (Buchberger et al., 2010). Multiple small-molecule-mediated conditional protein regulation systems have been proposed (Natsume and Kanemaki, 2017; Raina and Crews, 2010) and include the incorporation of destabilization domains from FK506 binding protein 12 (FKBP) (Banaszynski et al., 2006) (Stankunas et al., 2003), from E. coli-derived dihydrofolate reductase (ecDHFR) (Banaszynski et al., 2006; Iwamoto et al., 2010) or from human estrogen receptor ligand-binding domain (Miyazaki et al., 2012) that can be stabilized by the addition of shield-1 (FK506/tacrolimus), trimethoprim (TMP) or 4-hydroxytamoxifen, respectively.

Another strategy would be to incorporate the IKZF3 zinc finger-based degron tag that is responsive to immunomodulatory drugs (IMiDs) such as lenalidomide, pomalidomide or other thalidomide analogs including Cereblon E3 Ligase Modulation Drugs (CELMoDs) like Iberdomide. IMiDs can bind directly to the degron tag and recruit the cereblon ubiquitin ligase complex for polyubiquitination followed by degradation through the proteosomal machinery (Jan et al., 2021). A minimal IMiD-responsive IKZF3 degron has been mapped and shown to target heterologous proteins for destruction with IMiDs (Koduri et al., 2019). Similarly, sequences triggering auxin-induced degradation (Nishimura et al., 2009), ligand-induced degradation (Bonger et al., 2011), and small molecule-associated shutoff (Chung et al., 2015) or dTags (Nabet et al., 2018) have also been utilized for targeted protein degradation. Considering these advantages of specificity, reversibility and time required for depletion, methods of conditional protein depletion are advantageous to methods that attempt to control DNA or mRNA expression, and have been used in the context of CRISPR-Cas9-based gene editing (Maji et al., 2017) and synthetic immune receptors in adoptive cancer immunotherapy (Jan et al., 2021; Weber et al., 2021).

In the context of the SB transposon system, it was observed that SB transposase is sensitive to modifications and may lose transposase activity. In the past, many attempts have been made to create fusion variants of SB transposase for various applications but it was consensually observed that the fusion transposase always displayed reduced activity or even lost activity compared to the wild type transposase (Ivics et al., 2007; Kovac et al., 2020; Voigt et al., 2012; Yant et al., 2007). Methods to regulate SB transposase expression at the DNA level using the Tet-On system have been tested in the past but such methods are limited in that the Tet-On system is prone to leakiness and the temporal delay in terminating the presence of SB transposase protein in host cells (Cocchiarella et al., 2016).

### DESCRIPTION OF THE INVENTION

The inventors created novel SB transposase fusion proteins that can be conditionally regulated with regards to protein stability and transposition activity with pharmacologic agents and will be useful for pre-clinical and clinical applications in cell and gene therapy.

The inventors took on an approach to artificially instill control over SB transposase protein stability and transposition activity by fusing the SB transposase to either a degradation domain or a destabilizing domain. The inventors tested the IKZF3 zinc finger degron-tag as a degradation domain by fusing it to the SB transposase (degron-SB100X) and observed that the transposition activity of degron-SB100X could be regulated and even completely turned off in the presence of pomalidomide. Surprising and unexpectedly, the inventors found that pomalidomide interfered with the transposition activity of SB fusion transposase and also wild type SB transposase such that the effect of controlling transposition activity with the degron-SB100X fusion transposase was through controlling the stability of transposase protein and though direct inhibition of the transposition process. The degron-tag based SB fusion transposase and the use of pomalidomide and other IMiDs to control the stability and activity of SB fusion transposase are useful for pre-clinical and clinical applications in cell and gene therapy. The inventors also tested destabilizing domains, FKBP and ecDHFR, and observed, surprisingly and unexpectedly, that only the ecDHFR-based SB fusion transposase (dd-SB100X) could be stabilized and retained transposition activity in the presence of TMP, whereas expression of the FKBP-based SB fusion transposase could not be regulated. This behaviour was unexpected and non-obvious. The inventors characterized the ecDHFR-based SB fusion transposase (dd-SB100X) in detail. In summary, the structurally unstable protein referred to as destabilizing domain (dd) domain from E. coli dihydrofolate reductase (ecDHFR) was incorporated at the N-terminus of SB transposase, resulting in a fusion-transposase protein (dd-SB100X). ecDHFR destabilizing domain is largely unfolded and unstable when expressed in cells. The inventors show that this instability can be imparted to the SB transposase in the fusion transposase and induce rapid proteasome mediated SB fusion transposase protein degradation. The protein degradation can be prevented by trimethoprim (TMP) or using structurally similar ligands. TMP binds to and thereby, stabilizes the ecDHFR domain to prevent degradation of the fusion transposase. The inventors show that TMP can be used in a time and dose-dependent manner to control the stability of SB fusion transposase (dd-SB100X) and its transposition activity in human T cells. The inventors demonstrate that these novel SB fusion transposases allow rapid and precise control over SB transposase activity in host cells, exemplified in with several human cell lines and primary human T cells that are gene engineered to express a chimeric antigen receptor (CAR).

### BRIEF DESCRIPTION OF THE DRAWINGS

FIG. 1: Concept of SB fusion transposase proteins and validation of degron-SB100X fusion transposase in 293T cells and primary human T cells.

(1A) Schematic overview of SB fusion transposase variants. Each fusion construct contains a conditional protein regulation domain (grey box; varying lengths), a linker (white box with filled with dots; 42 bp) and full-length SB100X transposase (black box; 1020 bp).

(1B) Schematic overview of the small-molecule-induced conditional protein regulation systems tested in this study. (Left) Pomalidomide or other thalidomide derivatives recruit ubiquitin ligase to the fusion-transposase protein for rapid ubiquitination followed by degradation. (Middle) FK506 binding protein 12 (FKBP)-based destabilizing domain (dd) wherein the ligand (e.g. FK506) binds to and stabilizes the fusion-transposase protein. In the absence of ligand, the fusion-transposase protein is subjected to degradation. (Right) An ecDHFR-based fusion transposase protein, stabilized in the presence of its ligand (e.g. TMP, trimethoprim), and degraded in its absence.

(1C) Experimental workflow used for validating the SB fusion-transposase constructs for expression and activity. Briefly, 0.3×10⁶ Lenti-X 293T cells were seeded into 6 well plates and transfected with 3 μg of plasmid encoding the fusion-transpose, followed by incubation with respective ligands [trimethoprim (TMP) or FK506 or Pomalidomide (Poma)], or DMSO as control for 24 hours.

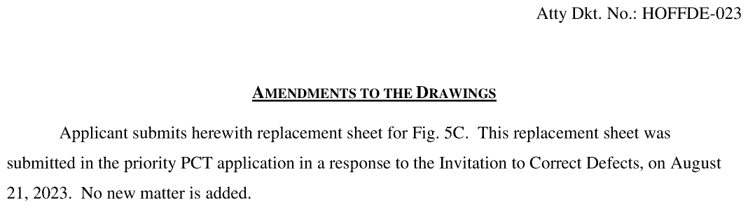

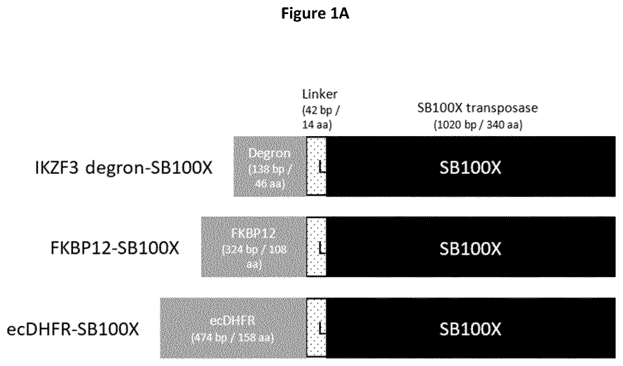

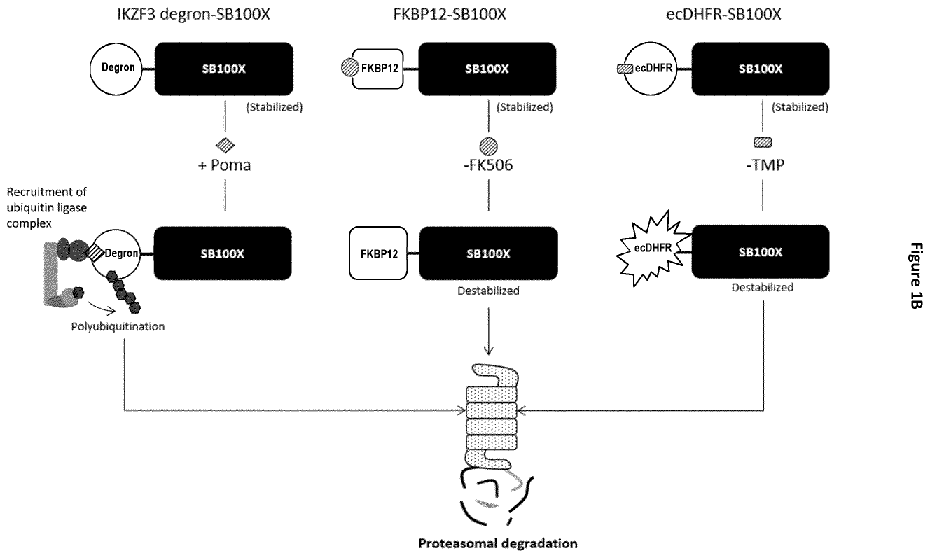

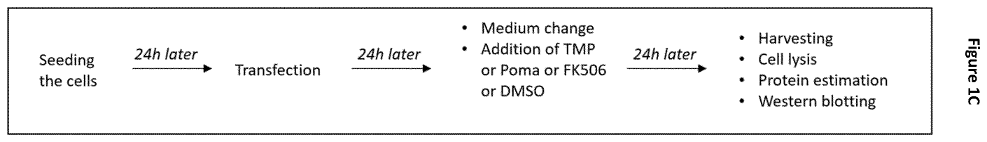

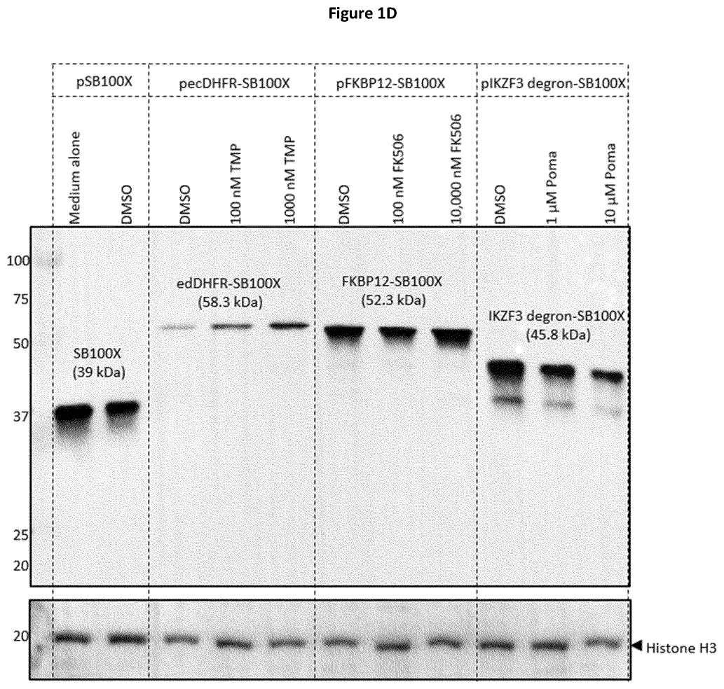

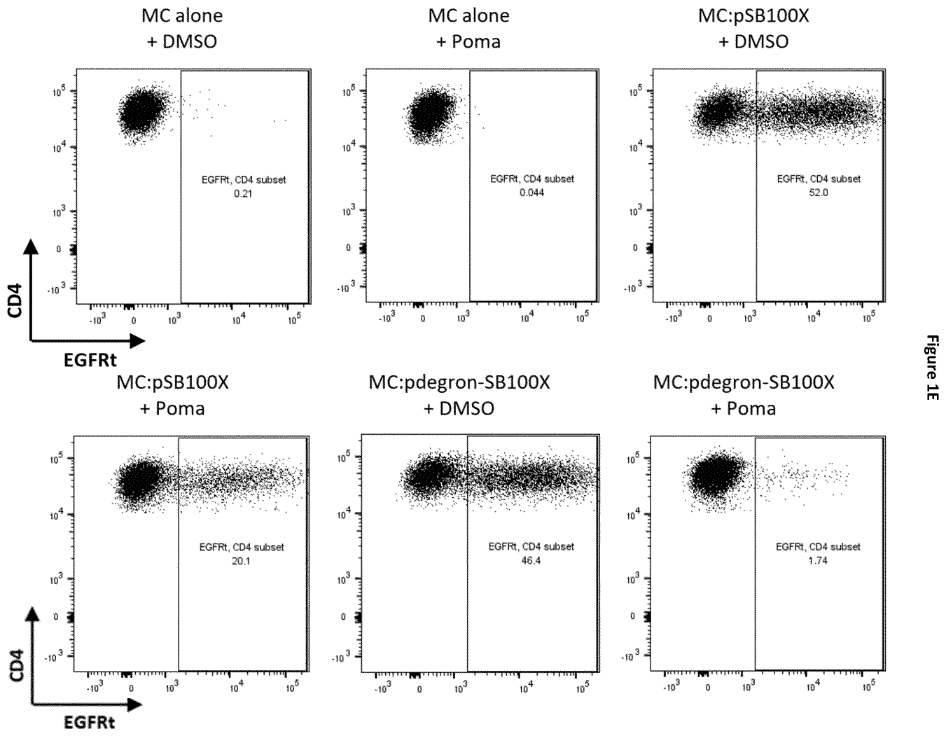

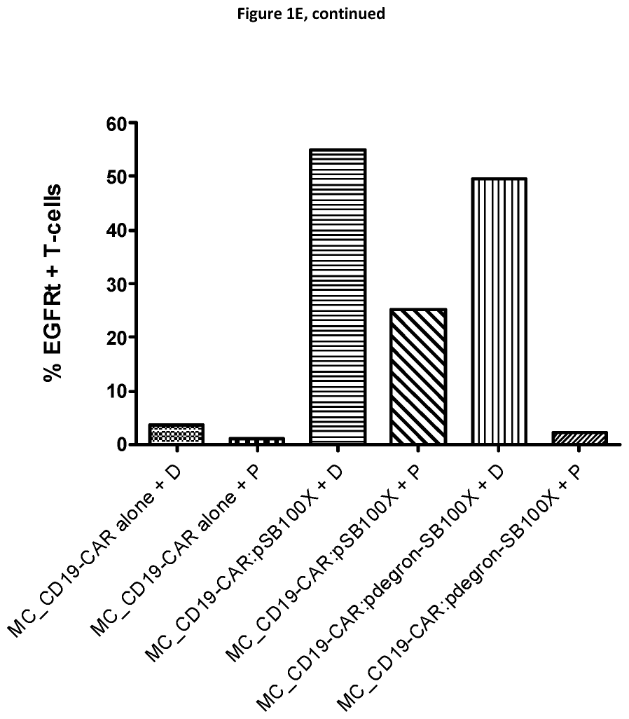

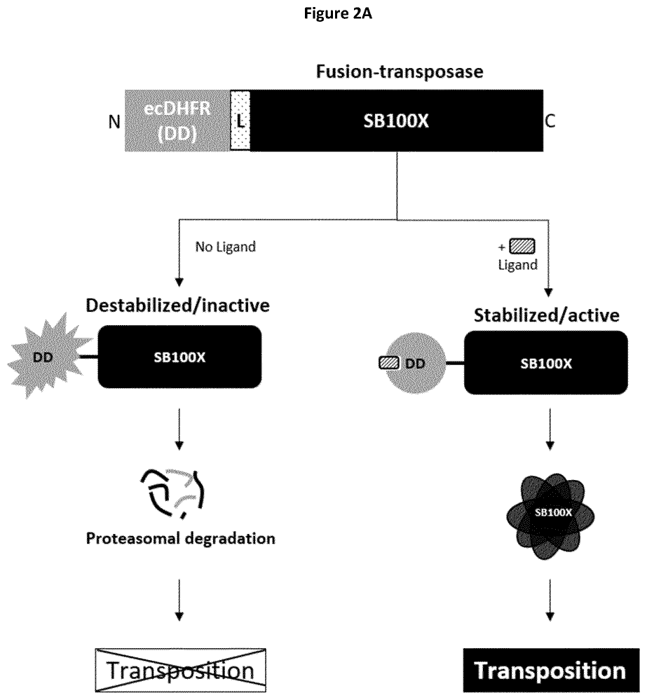

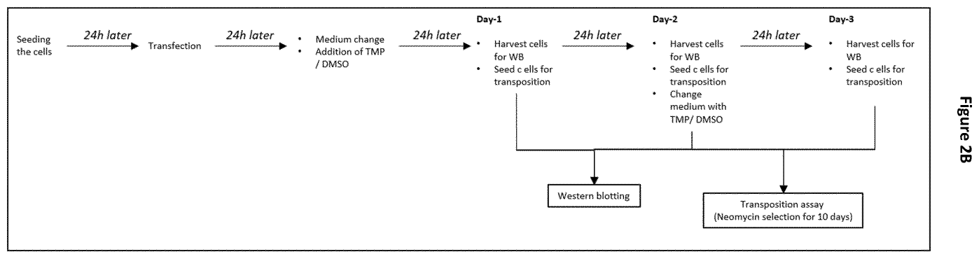

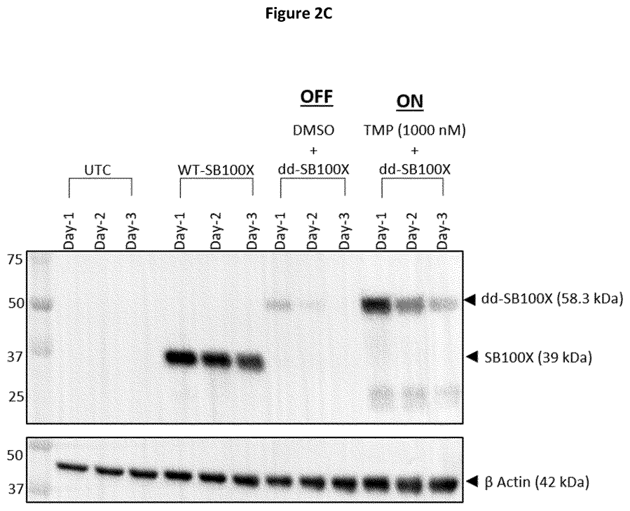

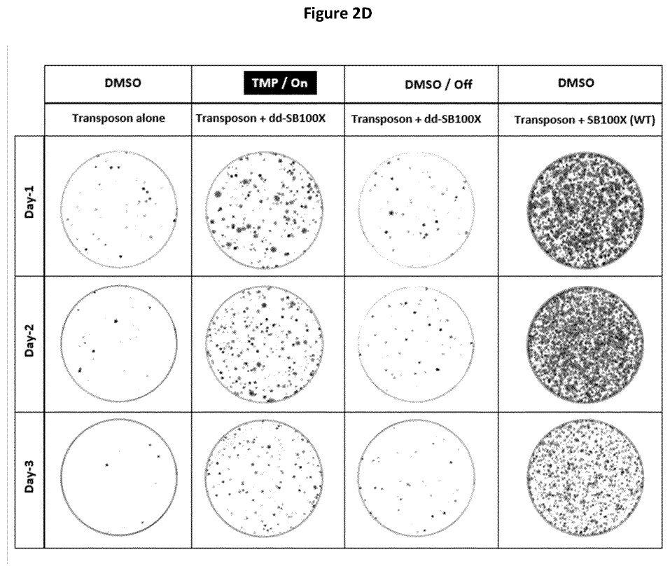

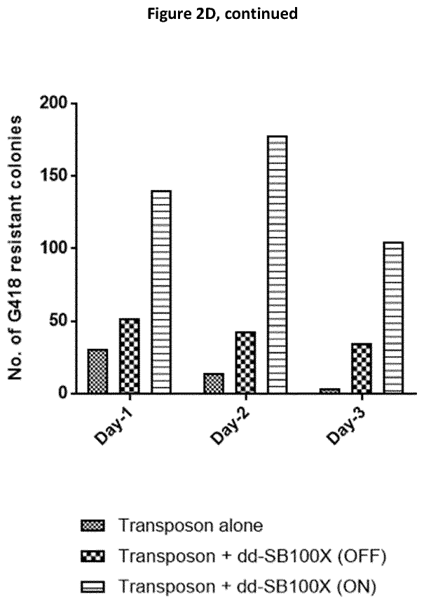

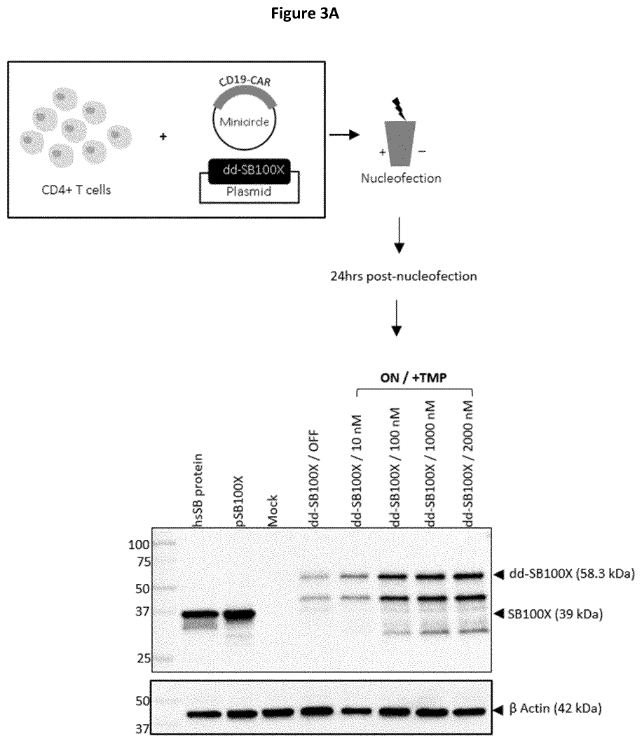

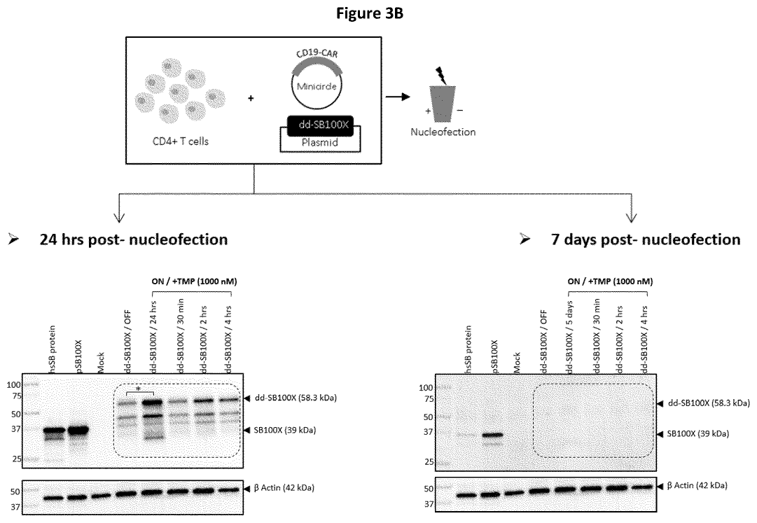

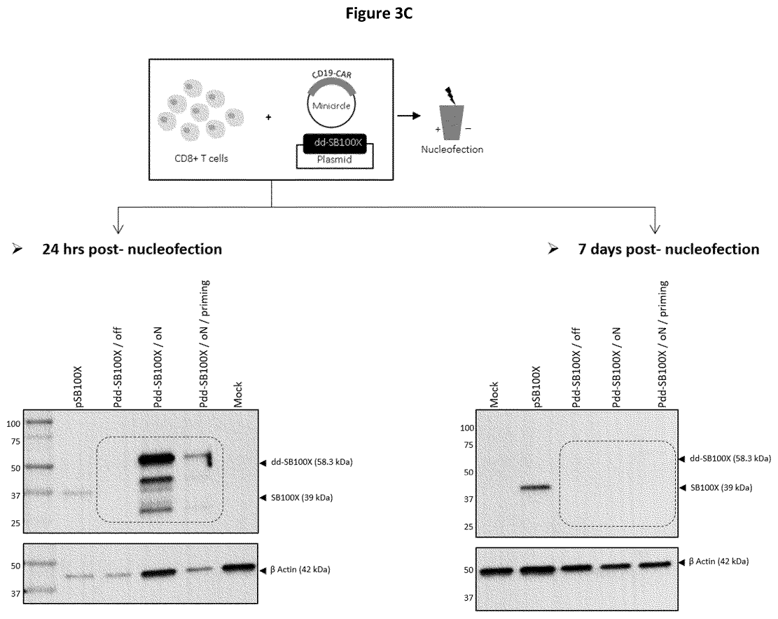

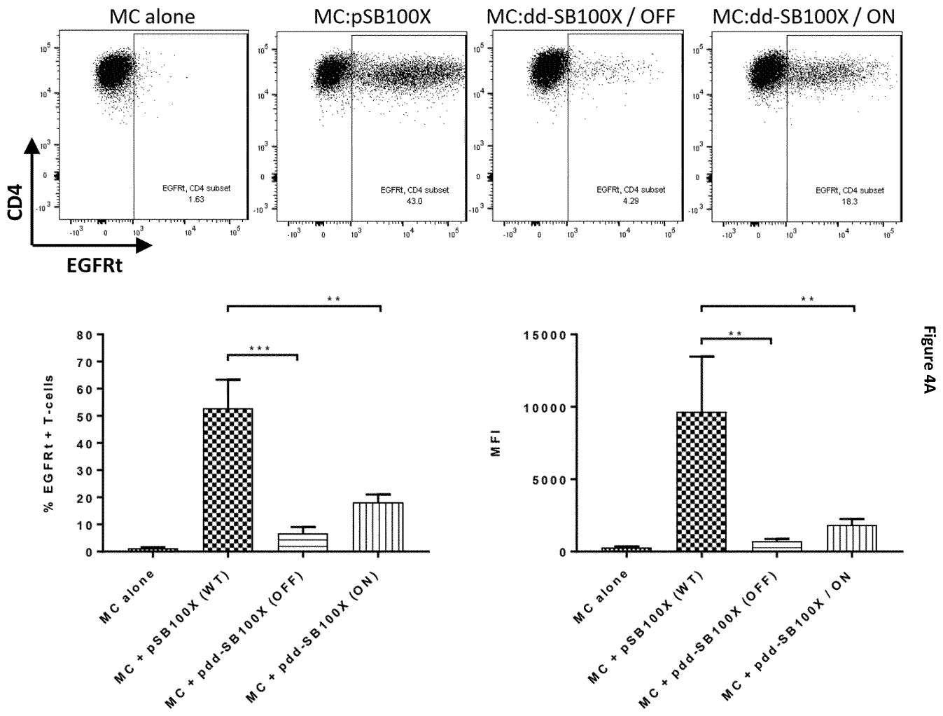

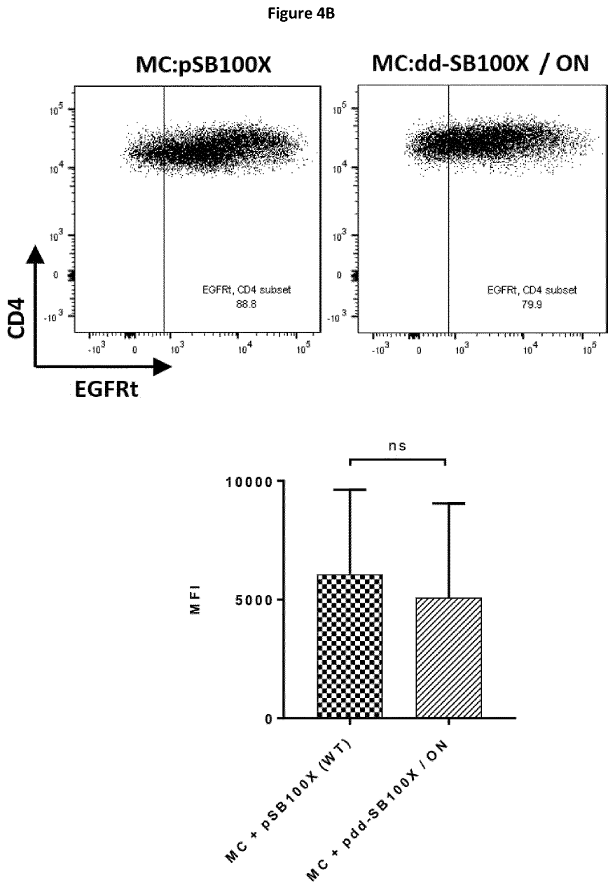

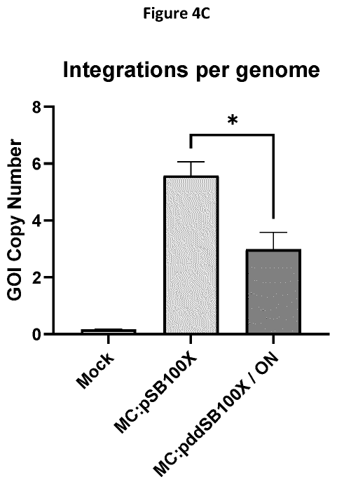

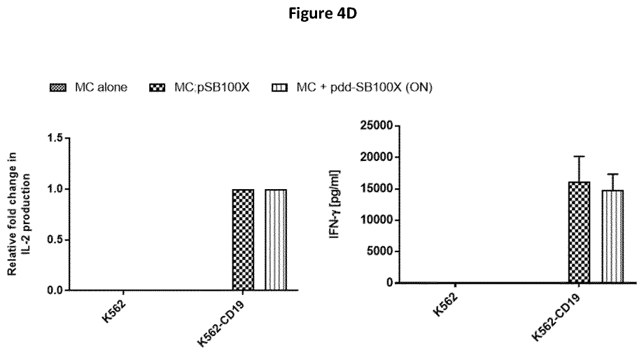

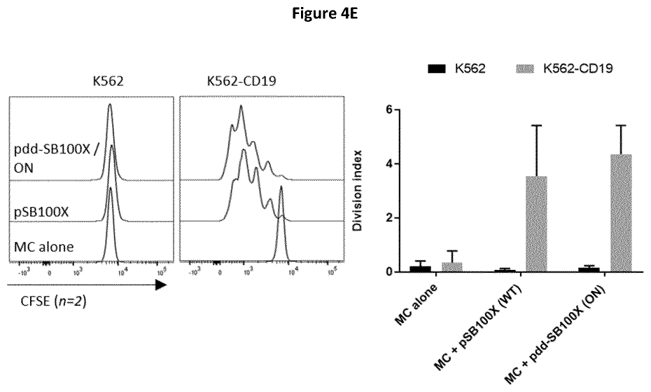

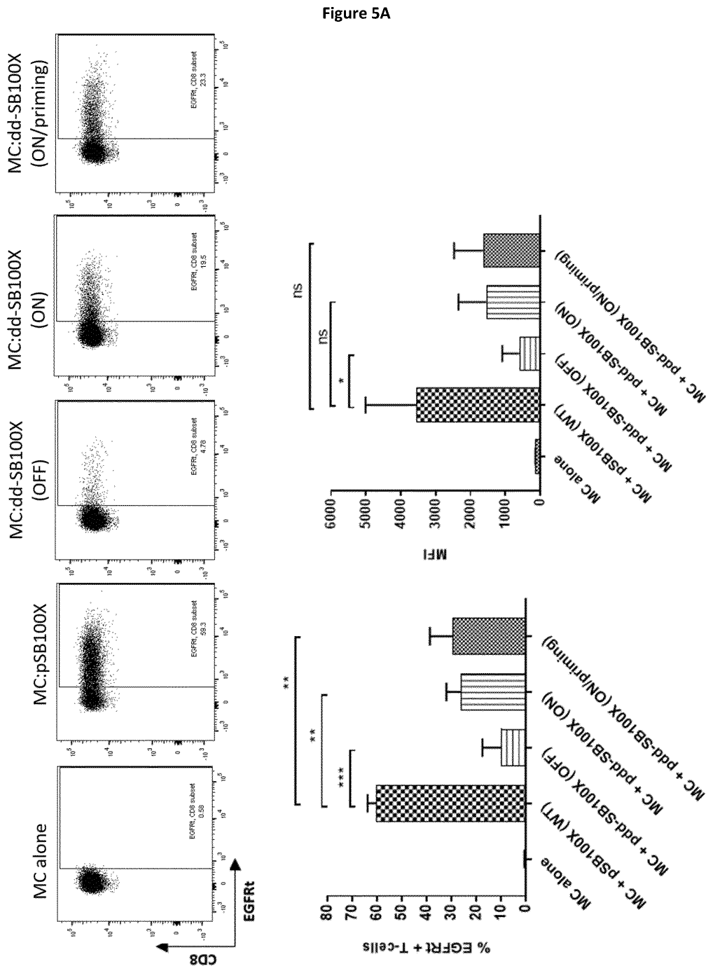

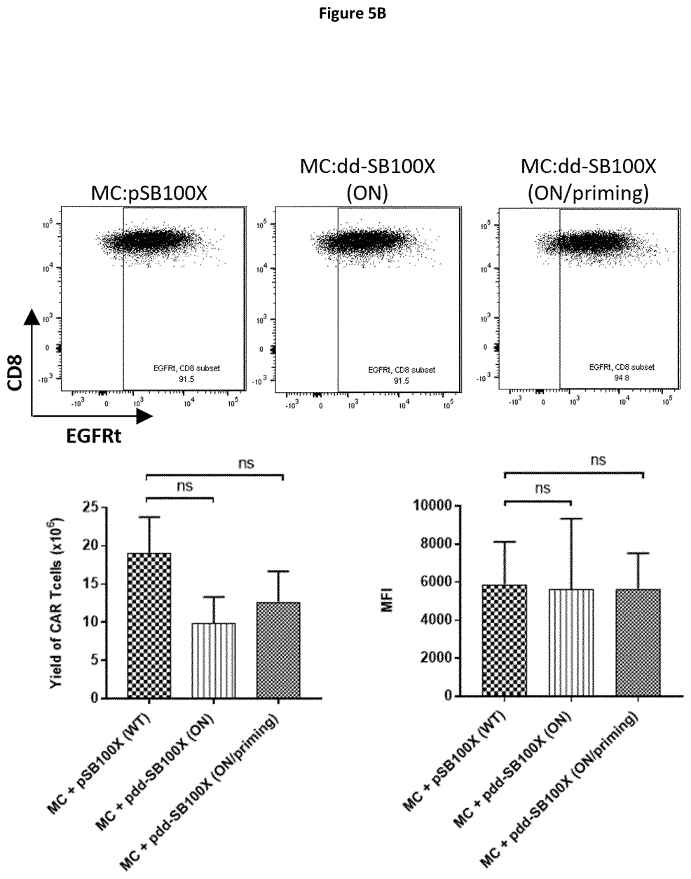

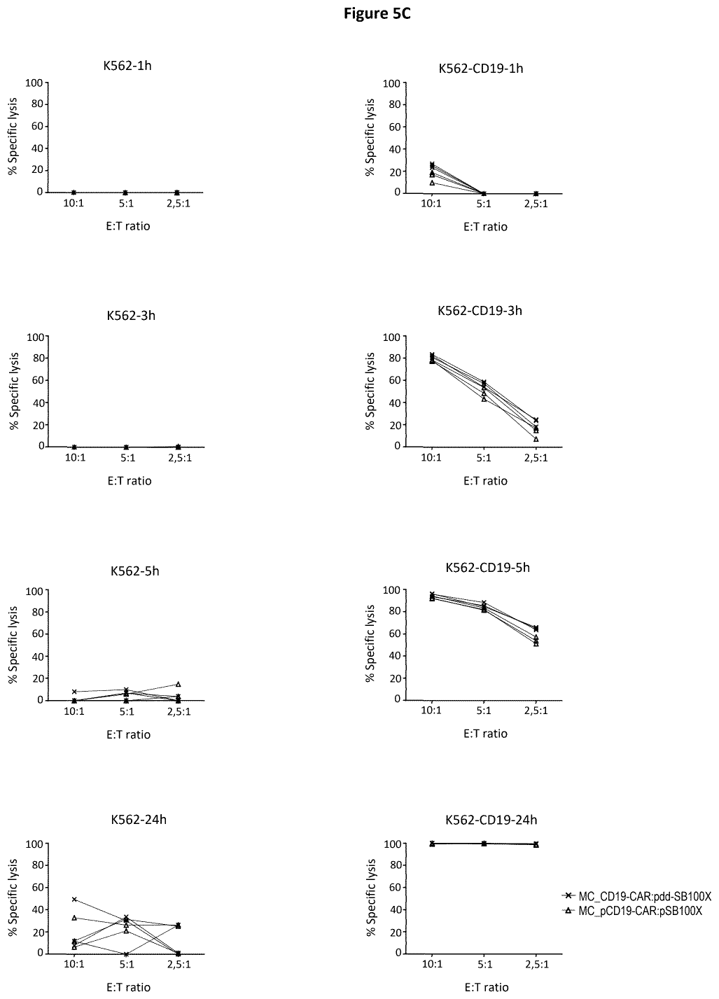

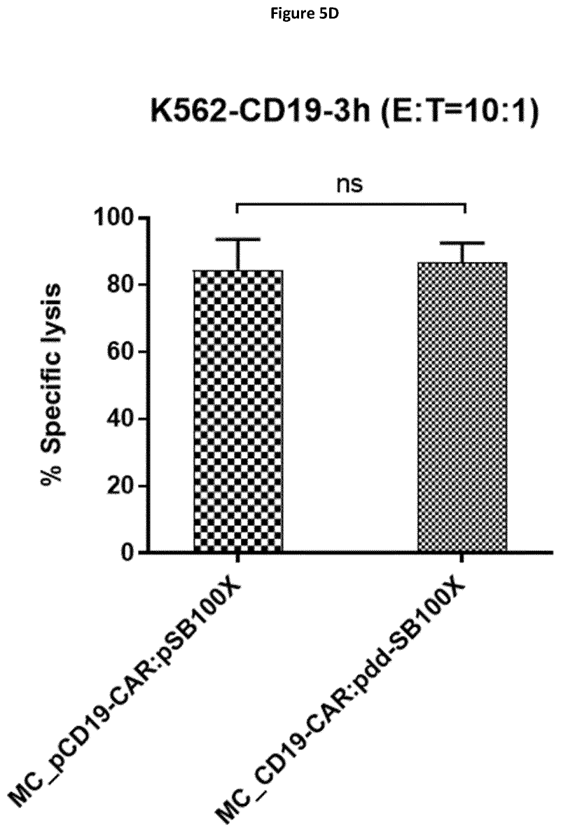

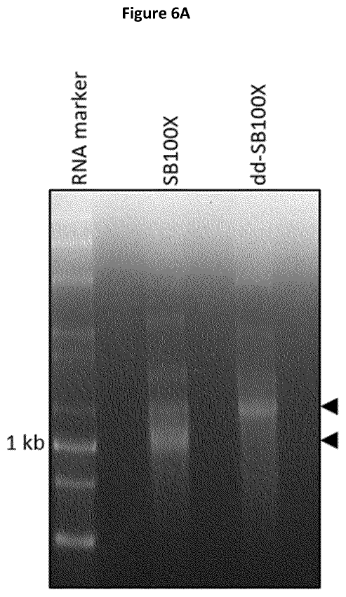

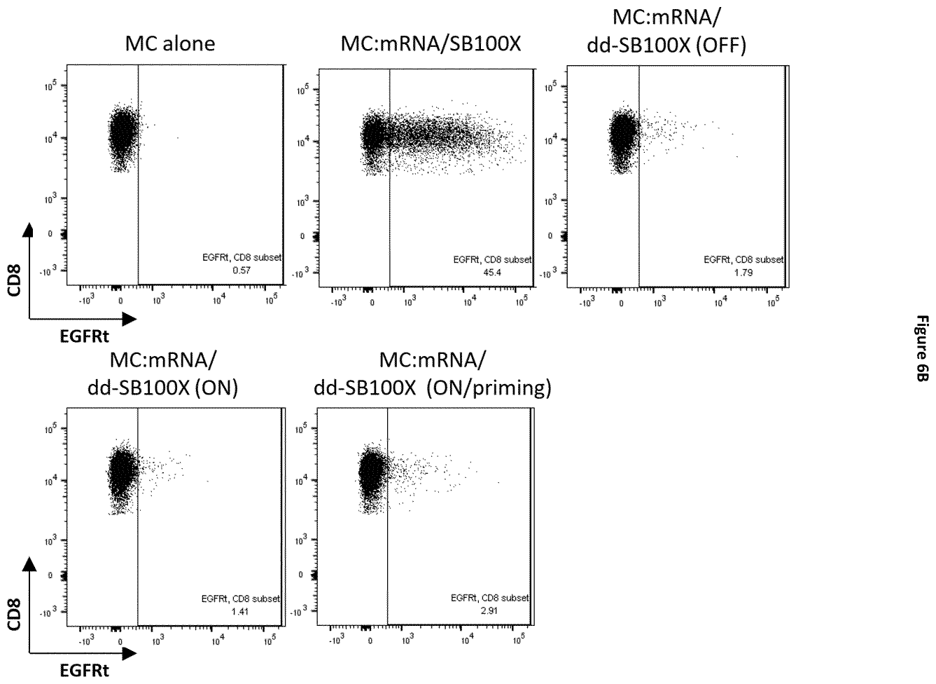

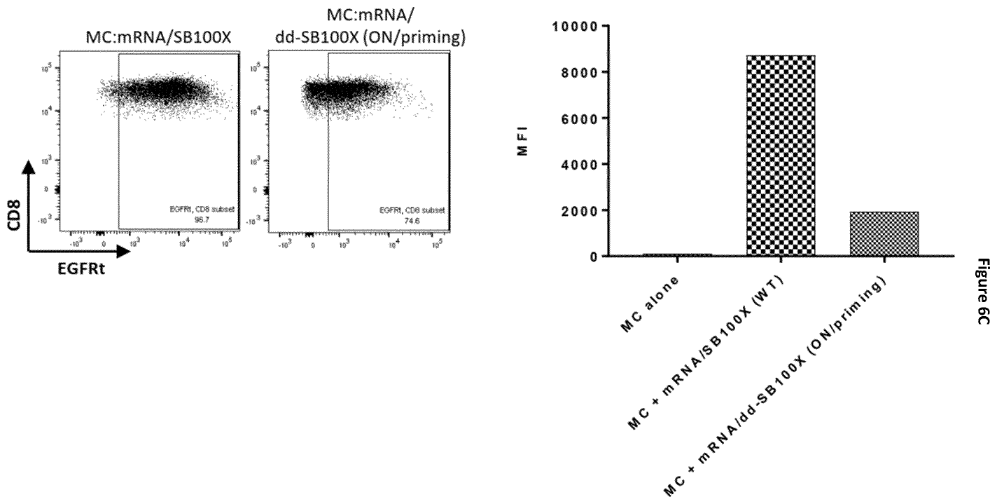

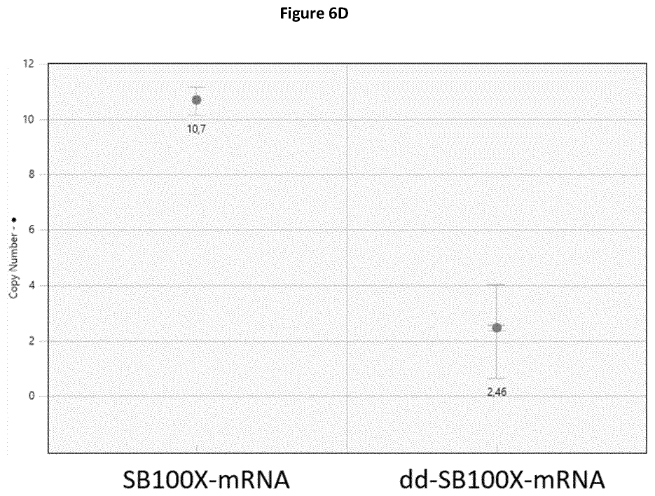

(1D) Analysis of SB fusion-transposase protein expression by Western blotting (continued from FIG. 1C). Cells were harvested; lysed and 6.6 μg of protein was subjected for immunoblot analysis using the anti-SB transposase antibody. The bottom panel shows the signal from Histone H3 as loading control that was detected using anti-Histone H3 antibody.

(1E) Testing the activity of IKZF3 zinc finger-based degron-SB100X fusion-transposase in human CD4+ T cells. For each condition, 2 million CD3/CD28 activated CD4+ T cells were nucleofected with minicircle DNA encoding a CD19-CAR_EGFRt SB transposon, and plasmid DNA encoding either the wild type SB transposase (SB100X) or the SB fusion-transposase (degron-SB100X). Cells were cultured either in the presence of Pomalidomide (P; 10 μM) or DMSO (D) at the time of nucleofection. At 7 days post-nucleofection, flow cytometric analysis was performed to determine the percentage of T cells that expresses the EGFRt reporter that was encoded in cis with the CD19-CAR. Top panel: Dot plots showing EGFRt expression on day 7 post-nucleofection. Bottom panel: Bar graph showing the percentage of EGFRt+ T cells on day 7 post-nucleofection (n=1).

FIG. 2: Expression and validation of dd-SB100X fusion transposase in HeLa cells.

(2A) Graphic illustrating the conditional destabilization of SB fusion-transposase dd-SB100X that uses a DHFR domain from E. coli (ecDHFR). The destabilized (misfolded) ecDHFR triggers rapid degradation of the SB fusion-transposase protein thereby inhibiting the transposition process. Trimethoprim (TMP) can be used to increase the stability of the ecDHFR; thereby stabilizing the SB fusion-transposase protein that can eventually perform transposition.

(2B) Experimental workflow used for validating the fusion-transposase construct for its protein expression and activity. Briefly, HeLa cells were transfected with the transposon and transposase plasmids, followed by incubation with TMP or DMSO for 1 to 3 days. Cells were harvested at different time points to assess protein expression by western blotting and assessing transposition activity.

(2C) Western blot analysis of to assess expression of dd-SB100X fusion-transposase protein. Transfected HeLa cells that were harvested at different time points (as shown in FIG. 2B), lysed and 15 μg of the protein from each sample was subjected for immunoblot analysis using anti-SB transposase antibody. The bottom panel shows the signal from β-actin as loading control that was detected using anti-β-actin antibody.

(2D) Transposition assay for evaluating the activity of dd-SB100X fusion-transposase. HeLa cells were transfected with neomycin-marked transposon plasmid and construct expressing wild type SB transposase (SB100X) or fusion-transposase (dd-SB100X), and selected for transposition events by antibiotic selection with G-418. Data representing the number of G-418-resistant colonies are shown on the right.

FIG. 3: Expression and validation of dd-SB100X fusion transposase in human T cells.

(3A) Dose dependent regulation of dd-SB100X fusion transposase expression by TMP in CD4+ T cells. For each condition, 2 million CD4+ T cells were nucleofected with CD19-CAR_EGFRt SB transposon minicircle DNA and plasmid encoding the fusion-transposase (dd-SB100X), and cultivated either in the absence ofTMP or in the presence ofTMP (range: 10 nM to 2000 nM) for 24 hours. As positive controls, CD4+ T cells were nucleofected with either CD19-CAR_EGFRt SB transposon minicircle DNA and plasmid encoding the wild type transposase (SB100X) or CD19-CAR_EGFRt SB transposon minicircle DNA and hsSB protein. At 24 hours post-nucleofection, cells were harvested; lysed and 10 μg of the protein from each sample was subjected for immunoblot analysis using anti-SB transposase antibody. The bottom panel shows the signal from β-actin as loading control that was detected using anti-β-actin antibody.

(3B) Time and dose-dependent regulation of dd-SB100X fusion transposase expression by TMP in CD4+ T cells. For each condition, 2 million CD4+ T cells were nucleofected with CD19-CAR_EGFRt SB transposon minicircle DNA and plasmid encoding the fusion-transposase (dd-SB100X), and were cultivated either in the absence of TMP or in the presence of TMP (1000 nM) for various time periods ranging from 30 min to 24 hours. As positive controls, CD4+ T cells were nucleofected with either CD19-CAR_EGFRt SB transposon minicircle DNA and plasmid encoding the wild type transposase (SB100X) or CD19-CAR_EGFRt SB transposon minicircle DNA and hsSB100X protein. For analyzing the protein levels, an aliquot of cells was taken from each of the reactions at 24 hours and 7-days post-nucleofection. Cells were harvested, lysed and 10 μg of the protein from each sample was subjected for immunoblot analysis using anti-SB transposase antibody. The bottom panels shows the signal from 3-actin as loading control that was detected using anti-β-actin antibody.

(3C) Validation of dd-SB100X fusion transposase in CD8+ T cells. For each condition, 2 million CD8+ T cells were nucleofected with CD19-CAR_EGFRt SB transposon minicircle DNA and plasmid encoding the fusion-transposase (dd-SB100X), and cultivated either in the absence of TMP or in the presence of TMP (1000 nM). CD8+ T cells were nucleofected with CD19-CAR_EGFRt SB transposon minicircle DNA and plasmid encoding the wild type transposase (SB100X) as a positive control, and with CD19-CAR_EGFRt SB transposon minicircle DNA alone as a negative control (mock). At 24 hours and at 7-days post-nucleofection, an aliquot of cells was harvested, lysed and 10 μg of protein from each sample was subjected for immunoblot analysis using anti-SB transposase antibody. The bottom panels shows the signal from β-actin as loading control that was detected using anti-β-actin antibody.

FIG. 4: Genomic and functional characterization of CAR T cells modified using SB fusion-transposase (dd-SB100X).

(4A) CAR gene transfer in CD4+ T cells using dd-SB100X fusion-transposase vs. wild type SB100X transposase. CD3/CD28 activated CD4+ T cells were nucleofected with CD19-CAR_EGFRt SB transposon minicircle DNA and plasmid encoding either the fusion-transposase (pdd-SB100X/dd-SB100X) or the wild type SB transposase (pSB100X/SB100X). At 7 days post-nucleofection, flow cytometric analysis was performed to determine the percentage of T cells that express the EGFRt reporter. Top panel: Dot plots show EGFRt expression on day 7 post-nucleofection. Bottom panel: bar diagram on the left shows the mean percentage ±s.d. of EGFRt+ T cells on day 7 post-nucleofection (n=3; P<0.05; 1-way ANOVA); bar diagram on the right shows the mean fluorescent intensity (MFI) for EGFRt based on the flow cytometric analysis (n=3; P<0.05; 1-way ANOVA).

(4B) Phenotype analysis of CD4+ CD19-CAR T cells after EGFRt enrichment. Engineered CAR T cells were cultured and enriched for EGFRt+ cells on day-9 post-nucleofection. Flow cytometric analysis was performed to determine the percentage of T cells that expresses the EGFRt reporter. Top panel: the dot plots show the EGFRt reporter expression after enrichment and expansion of EGFRt+ CD4+ T cells prior to functional testing. Bottom panel: the bar graph shows the mean fluorescent intensity (MFI) for EGFRt based on the flow cytometric analysis. The data was analyzed using Mann-Whitney test (P=0.6667) and the differences were statistically not significant (ns).

(4C) CAR Copy number analysis of CD8+ CD19 CAR T cells engineered with dd-SB100X fusion transposase. The number of integrations per genome was analyzed by ddPCR. Data shown are mean values of three biological replicates measured in technical triplicates and shown ±S.E.M. The data was analyzed using unpaired, two-tailed Student's T-test and differences were statistically significant (*, p=0.0274).

(4D) Cytokine production by CD4+ CD19 CAR-T cells engineered with dd-SB100X fusion transposase. ELISA was performed with supernatant obtained from triplicate wells after 24 hours of co-culture with K562/CD19 or K562 target cells (at an E:T ratio of 4:1). Data shown are mean values ±s.d. of one representative experiment.

(4E) Antigen specific proliferation by CD4+ CD19 CAR T cells engineered with dd-SB100X fusion transposase. Proliferation of CD4+ CAR T cells was examined by CFSE dye dilution after 72 hours of co-culture with K562/CD19 or K562 target cells. For analysis, triplicate wells were pooled and the proliferation of viable 7-AAD-negative T cells was analyzed. The division index (average number of cell divisions) was calculated using using FlowJo software. Data shown are mean values ±s.d. of one representative experiment.

FIG. 5: Priming T cells with TMP prior to gene transfer with SB fusion-transposase (dd-SB100X).

(5A) Priming T cells with TMP before nucleofection. CD8+ T cells were isolated from healthy donor PBMC and activated with CD3/CD28 bead stimulation. One day later, T cells were primed with TMP (1000 nM) for 24 hours before nucleofection. Cells were nucleofected with CD19-CAR_EGFRt SB transposon minicircle DNA and plasmid encoding either the wild type SB transposase (SB100X) or the fusion-transposase (dd-SB100X). Cells that were nucleofected with fusion-transposase (dd-SB100X) were immediately incubated either in the absence of TMP or in the presence of TMP (1000 nM). At 7 days post-nucleofection, flow cytometric analysis was performed to determine the percentage of T cells that expresses the EGFRt reporter. Top panel: Dot plots show EGFRt expression on day 7 post-nucleofection. Bottom panel: Bar diagram on the left shows the mean percentage ±s.d. of EGFRt+ T cells on day 7 post-nucleofection (n=3; P<0.05; 1-way ANOVA); bar diagram on the right shows the mean fluorescent intensity (MFI) of EGFRt based on the flow cytometric analysis.

(5B) Phenotype and yield of CD8+ CD19-CAR T cells engineered with dd-SB100X fusion transposase. T cells were enriched for EGFRt+ cells on day 9 post-nucleofection. Flow cytometry was performed to determine the percentage of T cells that expresses the EGFRt reporter and the MFI of EGFRt expression. Top panel: Dot plots show the EGFRt reporter expression after enrichment and expansion of EGFRt+ CD8+ T cells. Bottom panel: the bar graph on the left shows the yield of CAR T cells that was obtained within 14 days of culture following nucleofection with 2 million CD8+ T cells as input. The yield was calculated from absolute number of viable T cells (n=2 experiments); the bar diagram on the right shows the mean fluorescent intensity (MFI) of EGFRt based on the flow cytometric analysis.

(5C) Specific cytolytic activity of CD8+ CD19 CAR-T cells against K562/CD19 target cells. Cytolytic activity of CD8+ CD19-CAR T cells was evaluated by co-culture with K562/CD19 or K562 target cells using a bioluminescence-based assay. The assay was setup with effector T cells (CD8+ CD19-CAR T cells) at various effector/target (E:T) ratios in 96 well flat bottom plates. Luminescence intensities were recorded at specific time points (1, 3, 5, 24 hours) using a luminometer. Specific lysis was calculated using the standard formula. Data shown are from one representative experiment.

(5D) Specific cytolytic activity of CD8+ CD19 CAR-T cells engineered with dd-SB100X fusion transposase vs. wild type SB100X transposase. The bar graph shows the specific cytolytic activity against K562/CD19 target cells at an E:T ratio of 10:1 at the 3 hour time point from two independent experiments (n=2). Data was analyzed by unpaired t-test.

FIG. 6: mRNA as a source of fusion-transposase (dd-SB100X).

(6A) Analysis of in vitro transcribed mRNA by agarose gel electrophoresis. mRNA encoding wild type SB100X or the fusion-transposase (dd-SB100X) was synthesized using the respective plasmids and the mMessage mMachine T7 Ultra kit. For visualization, RNA marker and approximately around 1 μg of the mRNA from each sample was loaded onto an 1% agarose gel.

(6B) Activity of dd-SB100X fusion-transposase mRNA in CD8+ T cells. CD8+ T cells were isolated from healthy donor PBMCs and activated with CD3/CD28 bead stimulation. One day later, the activated CD8+ T cells were primed with TMP (1000 nM) for 24 hours before nucleofection. T Cells were nucleofected with CD19-CAR_EGFRt SB transposon minicircle DNA and mRNA encoding either the wild type SB transposase (SB100X) or the fusion-transposase (dd-SB100X). Post-nucleofection, fusion-transposase (dd-SB100X) nucleofected cells were immediately incubated either in the presence of TMP (1000 nM) or the absence of TMP. At 7 days post-nucleofection, flow cytometric analysis was performed to determine the percentage of T cells that expresses the EGFRt reporter. Dot plots show EGFRt expression on day 7 post-nucleofection. Data shown from one representative experiment (n=1).

(6C) Phenotype analysis of CD8+ CD19 CAR T cells after EGFRt enrichment. Engineered CAR T cells were enriched for EGFRt+ cells on day-9 post-nucleofection. Flow cytometric analysis was performed to determine the percentage of T cells that expresses the EGFRt reporter that is encoded in cis with the CD19-CAR. Dot plots shows EGFRt reporter expression after enrichment and expansion of EGFRt+ CD8+ T cells. The bar graph on the right show the mean fluorescent intensity (MFI) for EGFRt that was calculated based on the flow cytometric analysis.

(6D) Transposon copy number analysis in EGFRt+ CD8+ T cells that were nucleofected with CD19-CAR_EGFRt SB transposon minicircle DNA and mRNA encoding either the wild type SB transposase (SB100X) or the fusion-transposase (dd-SB100X) assessed by digital droplet PCR.

### DETAILED DESCRIPTION OF THE INVENTION

Sleeping Beauty (SB) transposase is a stable protein with a long half-life. Methods to regulate the activity and stability of SB transposase at protein level are currently lacking. There is a need in the clinic and art for such much methods, which are highly desirable. Fine-tuned control of transposase protein levels is very essential and important, as high amount of transposase, e.g., SB, are cytotoxic to the cells and lead to genotoxicity due to high transposon copy number and transposon remobilization. Therefore, rapid depletion of the transposase protein, e.g., SB transposase, after a desired genomic integration is valuable to prevent such toxicities. To overcome these challenges and limitations, the inventors developed conditional control systems by which the activity can be controlled and the protein stability of a transposase, e.g., SB transposase, can be perturbed using small molecules. In the current invention, the inventors screened and validated different small molecule mediated conditional protein regulation systems that are available and found that this it is not an obvious or a straight forward approach, because, unexpectedly, not all the protein regulation systems work in the case of transposon systems such as SB. After validation, the inventors further selected the best in class that works in the context of an SB transposon system that will be of great value in cell and gene therapy applications. The inventors created novel fusion-transposase variants—(i) by fusing the IKZF3 zinc finger degron-tag to the N terminus of SB transposase (degron-SB100X). In the presence of pomalidomide, there is a significant reduction in transposition activity, resulting from degradation of degron-SB100X and interference of pomalidomide with the transposition process; and (ii) by fusing the destabilizing domain from E. coli dihydrofolate reductase (ecDHFR) to the N terminus of SB transposase (dd-SB100X) so that instability is imparted to the fusion-transposase resulting in rapid degradation. This can be reversed by the use of cell permeable small molecules like trimetheoprim (TMP; a clinically available antibiotic) that binds and stabilizes the unfolded destabilizing domain so that the fusion-transposase protein is spared from protein degradation; and (iii) by fusing FKBP12 to the N terminus of SB transposase (FKBP-SB100X) which did however not lead to protein destabilization and demonstrates that the development and ensuing function of SB transposase fusion proteins is not predictable and not obvious. In particular, this invention provides a novel, functionally active, fusion-transposase that has a short-half-life whose stability and activity can be controlled by the use of TMP. Using this approach, the inventors demonstrate that the temporal kinetic and extent of transposition can be controlled and the resulting gene transfer rate and transposon copy number be steered. The invention is valuable and can be adapted to the clinical manufacturing of CAR T cells and other genetically engineered immune cell products. This invention enables virus-free, rapid and highly scalable generation of CAR T cells as well as point-of-care manufacturing. Moreover, this approach and method will also be applicable to other transposon technologies like PiggyBac, Tol2, Frog Prince, TcBuster, Mos1 and Hellraiser.

### Items of the Disclosure

The present invention provides, inter alia, the following items:
1. A complex, comprising:
   A) a destabilizing domain or degron tag; and
   B) a second protein domain or protein having enzymatic activity,
   wherein the destabilizing domain or degron tag is capable of modulating the half-life of the complex and/or is capable of modulating the enzymatic activity of the complex, and wherein the complex is capable of binding to a signaling molecule or ligand.
2. The complex of item 1, wherein the complex is a fusion protein comprising the destabilizing domain or degron tag and the second protein domain or protein.
3. The complex of any one of items 1-2, wherein the second protein domain or protein is a transposase domain and the enzymatic activity is transposase activity.
4. The complex of any one of items 1-3, wherein the destabilizing domain or degron tag is selected from:
   a) a destabilizing domain from E. coli-derived dihydrofolate reductase (ecDHFR),
   b) a destabilization domain from FK506 binding protein 12 (FKBP), or
   d) a degron tag, optionally wherein the degron tag is an IKZF3 zinc finger degron tag.
5. The complex of any one of items 1-4, wherein the destabilizing domain or degron tag is linked to the second protein domain or protein at the N-terminus.
6. The complex of any one of items 1-5, wherein the complex is a fusion protein and further comprises a linker, wherein the destabilizing domain or degron tag is linked to the second protein domain or protein via a linker.
7. The complex of any one of items 1-6, wherein the destabilizing domain or degron tag modulates the half-life of the complex when exposed to one or more signaling molecule(s) or ligand(s), optionally wherein the destabilizing domain or degron tag reduces the half-life of the complex compared to the half-life of the second protein domain or protein when not in complex with the destabilizing domain or degron tag.
8. The complex of any one of items 1-7, wherein the destabilizing domain or degron tag modulates the half-life of the complex when exposed to one or more signaling molecule(s) or ligand(s), optionally wherein the half-life of the complex is increased when the complex is exposed to the one or more signaling molecule(s) or ligand(s) compared to the same complex in absence of the one or more signaling molecule(s) or ligand(s).
9. The complex of any one of items 1-8, wherein the destabilizing domain or degron tag modulates the enzymatic activity of the complex.
10. The complex of any one of items 1-9, wherein the destabilizing domain or degron tag modulates the enzymatic activity of the complex when exposed to one or more signaling molecule(s) or ligand(s), optionally wherein the enzymatic activity of the complex is reduced when the complex is exposed to the one or more signaling molecule(s) or ligand(s) compared to the same complex in absence of the one or more signaling molecule(s) or ligand(s).
11. The complex of any one of items 1-10, wherein the one or more signaling molecule(s) or ligand(s) is/are selected from:
   a) immunomodulatory imide drugs,
   b) pomalidomide, lenalidomide, or thalidomide derivatives
   b) trimtheoprim,
   c) shield-1 or FK506/tacrolimus.
12. The complex of any one of items 1-11, wherein the one or more signaling molecule(s) or ligand(s) comprises or is pomalidomide.
13. The complex of any one of items 1-12, wherein the one or more signaling molecule(s) or ligand(s) comprises or is trimtheoprim.
14. The complex of any one of items 1-13, wherein the one or more signaling molecule(s) or ligand(s) reduces the enzymatic activity of the complex by at least about 10%, at least about 20%, at least about 30%, at least about 40%, at least about 50%, at least about 60%, at least about 70%, at least about 80%, at least about 90%, at least about 95%, at least about 98%, or at least about 99% compared to the same complex under the same physiological conditions in the absence of the ligand.
15. The complex of any one of items 1-14, wherein in the presence of the one or more signaling molecule(s) or ligand(s), the residual enzymatic activity of the complex is less than 10%, less than 9%, less than 8%, less than 7%, less than 6%, less than 5%, less than 4%, less than 3%, less than 2%, or less than 1% of the activity of the same complex in the absence of the one or more signaling molecule(s) or ligand(s).
16. The complex of any one of items 1-15, wherein the complex is destabilized in the absence of the one or more signaling molecule(s) or ligand(s), such that the half-life of the complex is reduced by at least about 10%, at least about 20%, at least about 30%, at least about 40%, at least about 50%, at least about 60%, at least about 70%, at least about 80%, at least about 90%, at least about 95%, at least about 98%, or at least about 99% when compared to the same complex in the presence of the ligand.
17. The complex of any one of items 1-16, wherein contacting the complex with the one or more signaling molecule(s) or ligand(s) stabilizes the complex, such that the half-life of the complex is restored to at least about 10%, at least about 20%, at least about 30%, at least about 40%, at least about 50%, at least about 60%, at least about 70%, at least about 80%, at least about 90%, at least about 95%, at least about 98%, or at least about 99% of the half-life of the same complex when not in contact with the ligand.
18. The complex of any one of items 1-17, wherein the second protein domain or protein is a transposase domain is or is derived from Sleeping Beauty, PiggyBac, Tol2, Frog Prince, TcBuster, Mos1, or Hellraiser.
19. The complex of any one of items 1-18, wherein the second protein domain or protein is a transposase domain from Sleeping Beauty or a transposase domain from Sleeping Beauty, optionally wherein the transposase domain is SB100X of wild type Sleeping Beauty or is derived from SB100X.
20. The complex of any one of items 1-19, wherein:
   a) the transposase domain is SB100X of wild-type Sleeping Beauty, the destabilizing domain or degron tag is a destabilizing domain from E. coli-derived dihydrofolate reductase (ecDHFR), optionally wherein the one or more signaling molecule(s) or ligand(s) comprises or is trimtheoprim (TMP); or
   b) the transposase domain is SB100X of wild-type Sleeping Beauty, the destabilizing domain or degron tag is is an IKZF3 zinc finger degron tag, optionally wherein the one or more signaling molecule(s) or ligand(s) comprises or is pomalidomide.
21. The complex of any one of items 1-20, wherein the complex is a fusion protein and comprises an amino acid sequence having at least 70%, at least 80%, at least 85%, at least 90%, at least 95%, at least 98%, or at least 99% sequence identity to an amino acid sequence encoded by the nucleotide sequence of any one of SEQ ID NOs: 1-3.
22. The complex of any one of items 1-20, wherein the complex is a fusion protein and wherein the fusion protein comprises or consists of the amino acid sequence encoded by the nucleotide sequence of any one of SEQ ID NOs: 1-3.
23. A nucleic acid, encoding the complex or components of the complex as defined in any one of items 1-22.
24. The nucleic acid of item 23, wherein the nucleic acid is an mRNA.
25. A composition or pharmaceutical composition, comprising the complex of nucleic acid encoding as defined in any one of items 1-24.
26. A method of modulating the half-life of a complex, wherein the complex is as defined in any one of items 1-22,
   the method comprising linking the second protein domain or protein to a destabilizing domain or a degron tag,
   wherein the destabilizing domain or degron tag is capable of modulating the half-life of the complex,
   wherein the destabilizing domain or degron tag is as defined in any one of items 1-22, wherein the second protein or protein domain is as defined in any one of items 1-22.
27. The method of item 26, further comprising contacting the complex with one or more signaling molecule(s) or ligand(s) as defined in any one of items 1-22.
28. A method of modulating the enzymatic activity of a complex,
   wherein the complex is as defined in any one of items 1-22,
   the method comprising linking the second protein domain or protein to a destabilizing domain or a degron tag,
   wherein the destabilizing domain or degron tag is capable of modulating the enzymatic of the complex,
   wherein the destabilizing domain or degron tag is as defined in any one of items 1-22,
   wherein the second protein or protein domain is as defined in any one of claims 1-22.
29. The method of items 28, further comprising contacting the complex with one or more signaling molecule(s) or ligand(s) as defined in any one of items 1-22.
30. A method for producing a genetically engineered cell,
   the method comprising contacting a cell of interest with a complex as defined in any one of items 1-22 or with a nucleic acid encoding the fusion protein as defined in any one of items 23-24.
31. The method of item 30, further comprising contact the cell with a donor.
32. The method of item 31, wherein the donor is a transposon donor that is a transposable element comprising a genetic cargo to be delivered to the cell.
33. The method of item 32, wherein the transposon donor is a plasmid or a minicircle DNA.
34. The method of any one of items 30-33, wherein the genetic cargo comprises a chimeric antigen receptor (CAR).
35. The method of any one of items 30-34, wherein the method is limited to a total maximum time period from contacting the cells of interest to obtaining the final genetically engineered cell product of 7 days, 6 days, 5 days, 4 days, 3 days, 2 days, or 1 day.
36. A genetically engineered cell obtained by the method as defined in any one of items 30-35.
37. The genetically engineered cell as defined in item 36 for use in a method of treating a disease.
38. The genetically engineered cells for use of item 37, wherein the cell is an immune cell and wherein the disease is cancer.
39. The genetically engineered cell for use of item 38, wherein the immune cell is a T cell and the genetic cargo delivered to the T cell is a chimeric antigen receptor (CAR) targeting a surface antigen expressed by the cancer.
40. The genetically engineered cell of any one of items 36-39, wherein the cell comprises 10, 9, 8, 7, 6, 5 or fewer than 5 copies of the genetic cargo integrated into its genome.
41. The genetically engineered cell of item 40, wherein the cell comprises no more than 5 copies of the genetic cargo integrated into its genome.
42. A method for treatment, comprising a step of obtaining cells from a patient to thereby isolate the cells, contacting the isolated patient cells ex vivo with the complex, as defined in any one of items 1-22 and a donor, as defined in any one of items 31-34, to deliver genetic cargo to the patient cells, and administering the resulting genetically engineered cells to the patient, thereby treating the patient.
43. The method of item 42, wherein the method further comprises contacting the complex and the patient cells ex vivo with one or more signaling molecule(s) or ligand(s) as defined in any one of items 1-22.
   Destabilizing Domains and Complexes Thereof with Further Proteins or Protein Domains

Destabilizing domains (DDs) are known in the art. Destabilizing domains are protein sequences which are inherently unstable under physiological conditions, and thus by association with an otherwise stable second protein can confer a reduction in the half-life under physiological conditions of the resulting complex of the destabilizing domain and the second protein, compared to the half-life of the second protein under the same physiological conditions when not associated with the destabilizing domains.

Destabilizing domains can typically by stabilized in a controlled and reversible manner by a signalling molecule, i.e., a ligand. Binding of the ligand to the destabilizing domain partially or fully stabilizes the domain and thereby increases the half-life of a complex of the destabilizing domain and a second protein under physiological conditions when compared to the half-life of the same complex under the same physiological conditions in the absence of the ligand.

In a preferred embodiment, the destabilizing domain is a destabilizing domain from E. coli-derived dihydrofolate reductase (ecDHFR), a destabilizing domain from FK506 binding protein 12 (FKBP), or a destabilizing domain from human estrogen receptor ligand-binding domain.

In one embodiment, the destabilizing domain is a destabilizing domain from a destabilizing domain from FK506 binding protein 12 (FKBP).

In one embodiment, the destabilizing domain is a destabilizing domain from human estrogen receptor ligand-binding domain.

In a more preferred embodiment, the destabilizing domain is a destabilizing domain from E. coli-derived dihydrofolate reductase (ecDHFR).

In a preferred embodiment, the ligand that can partially or fully stabilize the destabilizing domain is trimethoprim (TMP), shield-1, FK506/tacrolimus, or4-hydroxytamoxifen.

In one embodiment, the destabilizing domain is a destabilizing domain from a destabilizing domain from FK506 binding protein 12 (FKBP) and the ligand (i.e., signalling molecule) that fully or partially stabilizes the destabilizing domain is shield-i or FK506/tacrolimus.

In one embodiment, the destabilizing domain is a destabilizing domain from human estrogen receptor ligand-binding domain and the ligand (i.e., signalling molecule) that fully or partially stabilizes the destabilizing domain is 4-hydroxytamoxifen.

In a more preferred embodiment, the destabilizing domain is a destabilizing domain from E. coli-derived dihydrofolate reductase (ecDHFR) and the ligand (i.e., signalling molecule) that fully or partially stabilizes the destabilizing domain is trimethoprim (TMP).

In a preferred embodiment, the complex, e.g., preferably fusion protein, of the invention is destabilized in the absence of the ligand (i.e., signaling molecule) such that the half-life of the complex under physiological conditions is reduced by at least about 10%, at least about 20%, at least about 30%, at least about 40%, at least about 50%, at least about 60%, at least about 70%, at least about 80%, at least about 90%, at least about 95%, at least about 98%, or at least about 99% when compared to the same complex under the same physiological conditions in the presence of the ligand.

In a preferred embodiment, contacting the complex, e.g., preferably fusion protein, of the invention with the ligand (i.e., signaling molecule) stabilizes the complex such that the half-life of the complex under physiological conditions is restored to at least about 10%, at least about 20%, at least about 30%, at least about 40%, at least about 50%, at least about 60%, at least about 70%, at least about 80%, at least about 90%, at least about 95%, at least about 98%, or at least about 99% of the half-life of the same complex under the same physiological conditions when not in contact with the ligand.

In a preferred embodiment, the complex of a destabilizing domain and a second protein can be formed via a fusion protein, i.e., via an N-terminal or C-terminal fusion of the destabilizing domain to the second protein.

In a more preferred embodiment, the destabilizing domain is fused to the second protein at the N-terminus.

The second protein comprised in the complex (e.g., preferably a protein fusion) of the destabilizing domain and the second protein is not particularly limited in its molecular makeup and may itself comprise one or more different protein domains. In an embodiment, the second protein can be a fusion of two otherwise unrelated proteins or protein domains itself. In another embodiment, the second protein can be a fusion of more than two otherwise unrelated proteins or protein domains.

In an embodiment, the complex (e.g., preferably a protein fusion) may comprise more than one destabilizing domains. The one or more destabilizing domains may be the same or may be different.

In an embodiment, the complex (e.g., preferably a protein fusion) may, in addition to a destabilizing domain and a second protein further comprise a degron tag. Degron tags are known in the art and described in a separate section of the disclosure of the present invention.

In another preferred embodiment, the ligand (i.e., signalling molecule) that reduces the half-life of the complex, e.g., preferably fusion protein, of the present invention comprising a destabilizing tag and a second protein domain or protein is a Cereblon E3 Ligase Modulation Drug (CELMoDs). CELMoDs is a class of compounds known in the art and includes, for example, iberdomide, but is not limited thereto. In one embodiment, the ligand (i.e., signaling molecule) is iberdomide. In one embodiment, the ligand (i.e., signaling molecule) is a derivative from lenalidomide or pomalidomide. In one embodiment, the ligand (i.e., signaling molecule) is iberdomide CC-220 or CC-92480.

Degron Tags and Complexes Thereof with Further Proteins or Protein Domains

Degron tags are known in the art. Degron tags are protein sequences which are capable of regulating protein degradation, and thus by association with an otherwise stable second protein can confer a reduction in the half-life under physiological conditions of the resulting complex of the degron tag domain and the second protein, compared to the half-life of the second protein under the same physiological conditions when not associated with the degron tag.

Degron tags can be inducible, i.e., their function to trigger protein degradation may be controlled by a signalling molecule, i.e., a ligand. Binding of the ligand to the degron tag can cause degradation of the degron tag and the complex it is associated with, e.g., in the form a protein fusion, and thereby reduce the half-life of a complex of the degron tag and a second protein under physiological conditions when compared to the half-life of the same complex under the same physiological conditions in the absence of the ligand.

Under specific circumstances, the binding of a ligand to the degron tag may also cause other effects to a complex comprising the degron tag. For example, the interaction between the ligand and the complex comprising the degron tag and a second protein or protein domain may alter the activity of the complex that the second protein or protein domain confers.

In some cases, the binding of a ligand to the degron may primarily or exclusively work by altering the activity of the complex that the degron tag is comprised in, and not trigger protein degradation.

In a preferred embodiment, interaction between the ligand and the complex, e.g., preferably fusion protein, of the invention comprising a degron tag and a second protein alters the enzymatic activity of the complex conferred by the second protein without affecting the protein degradation rate and/or half-life of the complex under physiological conditions compared to the same complex under the same physiological conditions in the absence of the ligand (i.e., signalling molecule).

In a preferred embodiment, interaction of the ligand with the complex, e.g., preferably fusion protein, of the invention comprising a degron tag and a second protein having enzymatic activity reduces the enzymatic activity of the complex under physiological conditions compared to the enzymatic activity of the same complex under the same physiological conditions in the absence of the ligand (i.e., signaling molecule).

In a preferred embodiment, interaction of the ligand with the complex, e.g., preferably fusion protein, of the invention comprising a degron tag and a second protein having enzymatic activity reduces the enzymatic activity of the complex under physiological conditions compared to the enzymatic activity of the same complex under the same physiological conditions in the absence of the ligand (i.e., signaling molecule) but does not affect the protein degradation rate and/or half-life of the complex when compared to the protein degradation rate and/or half-life of the same complex under the same physiological conditions in the absence of the ligand (i.e., signaling molecule).

In a preferred embodiment, the degron tag is an IKZF3 zinc finger degron tag.

In a preferred embodiment, the ligand that reduces the enzymatic activity of the complex, e.g., preferably fusion protein, of the present invention comprising a degron tag and a second protein domain or protein is an immunomodulatory imide drug (IMiD). IMiDs is a class of compounds known in the art that generally encompasses thalidomide and its derivatives.

In a preferred embodiment, the ligand that reduces the enzymatic activity of the complex, e.g., preferably fusion protein, of the present invention comprising a degron tag and a second protein domain or protein is pomalidomide, lenalidomide, thalidomide, or a thalidomide analogue.

In a preferred embodiment, the ligand that reduces the enzymatic activity of the complex, e.g., preferably fusion protein, of the present invention comprising a degron tag and a second protein domain or protein is pomalidomide.

In a preferred embodiment, the degron tag comprised in the complex, e.g., preferably fusion protein, of the invention comprising a degron tag and a second protein domain or protein is an IKZF3 zinc finger degron tag and the second protein domain or protein is a transposase domain.

In a more preferred embodiment, the degron tag comprised in the complex, e.g., preferably fusion protein, of the invention comprising a degron tag and a second protein domain or protein is an IKZF3 zinc finger degron tag and the second protein domain or protein is a transposase domain from wild type Sleeping Beauty (SB100X).

In an even more preferred embodiment, the degron tag comprised in the complex, e.g., preferably fusion protein, of the invention comprising a degron tag and a second protein domain or protein is an IKZF3 zinc finger degron tag and the second protein domain or protein is a transposase domain from wild type Sleeping Beauty (SB100X), and the enzymatic activity (i.e., transposition activity) of the complex is reduced in the presence of a ligand (i.e., signaling molecule) under physiological conditions when compared to the same complex under the same physiological conditions in the absence of the ligand. In a preferred embodiment, the ligand (i.e., signaling molecule) that mediates the reduction of enzymatic activity (i.e., reduced transposition activity) of the complex, e.g., preferably protein fusion, of the IKZF3 zinc finger degron tag and the transposase domain from wild type Sleeping Beauty (SB100X) is an immunomodulatory imide drug (IMiD), thalidomide, or a thalidomide derivative. In an even more preferred embodiment, the ligand (i.e., signaling molecule) that mediates the reduction of enzymatic activity (i.e., reduced transposition activity) of the complex, e.g., preferably protein fusion, of the IKZF3 zinc finger degron tag and the transposase domain from wild type Sleeping Beauty (SB100X) is pomalidomide or lenalidomide. In yet an even more preferred embodiment, the ligand (i.e., signaling molecule) that mediates the reduction of enzymatic activity (i.e., reduced transposition activity) of the complex, e.g., preferably protein fusion, of the IKZF3 zinc finger degron tag and the transposase domain from wild type Sleeping Beauty (SB100X) is pomalidomide.

In a preferred embodiment, the ligand (i.e., signaling molecule) mediates a reduction of enzymatic activity (i.e., reduced transposition activity) of the complex, e.g., preferably protein fusion, of the IKZF3 zinc finger degron tag and the transposase domain from wild type Sleeping Beauty (SB100X) under physiological conditions by at least about 10%, at least about 20%, at least about 30%, at least about 40%, at least about 50%, at least about 60%, at least about 70%, at least about 80%, at least about 90%, at least about 95%, at least about 98%, or at least about 99% compared to the same complex under the same physiological conditions in the absence of the ligand. In a preferred embodiment, the ligand (i.e., signaling molecule) that mediates this reduction is pomalidomide.

In a preferred embodiment, in the presence of pomalidomide, the residual enzymatic activity (i.e., transposition activity) of the complex, e.g., preferably fusion protein, of the IKZF3 zinc finger degron tag and the transposase domain from wild type Sleeping Beauty (SB100X) of the invention under physiological conditions is less than 10%, less than 9%, less than 8%, less than 7%, less than 6%, less than 5%, less than 4%, less than 3%, less than 2%, or less than 1% of the activity of the same complex under the same physiological conditions in the absence of pomalidomide.

In a preferred embodiment, in the presence of pomalidomide, the residual enzymatic activity (i.e., transposition activity) of the complex, e.g., preferably fusion protein, of the IKZF3 zinc finger degron tag and the transposase domain from wild type Sleeping Beauty (SB100X) of the invention under physiological conditions is less than 10% of the activity of the same complex under the same physiological conditions in the absence of pomalidomide.

In a more preferred embodiment, in the presence of pomalidomide, the residual enzymatic activity (i.e., transposition activity) of the complex, e.g., preferably fusion protein, of the IKZF3 zinc finger degron tag and the transposase domain from wild type Sleeping Beauty (SB100X) of the invention under physiological conditions is less than 5% of the activity of the same complex under the same physiological conditions in the absence of pomalidomide.

In a more preferred embodiment, in the presence of pomalidomide, the residual enzymatic activity (i.e., transposition activity) of the complex, e.g., preferably fusion protein, of the IKZF3 zinc finger degron tag and the transposase domain from wild type Sleeping Beauty (SB100X) of the invention under physiological conditions is less than 2% of the activity of the same complex under the same physiological conditions in the absence of pomalidomide.

In a preferred embodiment, the protein degradation rate and/or half-life of the complex, e.g., preferably fusion protein, of the IKZF3 zinc finger degron tag and the transposase domain from wild type Sleeping Beauty (SB100X) of the invention under physiological conditions is not substantially affected by the presence of pomalidomide.

In a preferred embodiment, the protein degradation rate and/or half-life of the complex, e.g., preferably fusion protein, of the IKZF3 zinc finger degron tag and the transposase domain from wild type Sleeping Beauty (SB100X) of the invention under physiological conditions in the presence of pomalidomide is the same as that of the same complex under the same conditions in the absence of pomalidomide.

In a preferred embodiment, the complex of a degron tag and a second protein can be formed via a fusion protein, i.e., via an N-terminal or C-terminal fusion of the degron tag to the second protein.

In a more preferred embodiment, the degron tag is fused to the second protein at the N-terminus.

The second protein comprised in the complex (e.g., preferably a protein fusion) of the degron tag and the second protein is not particularly limited in its molecular makeup and may itself comprise one or more different protein domains. In an embodiment, the second protein can be a fusion of two otherwise unrelated proteins or protein domains itself. In another embodiment, the second protein can be a fusion of more than two otherwise unrelated proteins or protein domains.

In a preferred embodiment, the second protein or protein domain comprised in the complex, e.g., preferably fusion protein, of the present invention is a transpose domain that is capable of mediating transposition. Suitable transposases and transposase domains are known in the art.

Non-limiting examples of suitable transposase domains to be comprised in the complex of the invention are transposase domains from Sleeping Beauty, PiggyBac, Tol2, Frog Prince, TcBuster, Mos1, or Hellraiser.

In a preferred embodiment, the second protein domain or protein domain comprised in the complex, e.g., preferably fusion protein, of the invention is the transposase domain from wild type Sleeping Beauty (SB100X).

In another preferred embodiment, the second protein domain or protein domain comprised in the complex, e.g., preferably fusion protein, of the invention is the transposase domain from a Sleeping Beauty transposase that has higher or lower transposition activity compared to SB100X, and/or is a derivative of SB100X transposase.

In another preferred embodiment, the second protein domain or protein domain comprised in the complex, e.g., preferably fusion protein, of the invention is the unmodified transposase domain from the Sleeping Beauty transposase.

In another preferred embodiment, the second protein domain or protein domain comprised in the complex, e.g., preferably fusion protein, of the invention is the an enhanced transposase domain from the Sleeping Beauty transposase (SB10 or SB11).

In another preferred embodiment, the second protein domain or protein domain comprised in the complex, e.g., preferably fusion protein, of the invention is an hsSB transposase domain from from a Sleeping Beauty transposase, that has higher or lower transposition activity compared to SB100X, and/or has 99% or more sequence identity with SB100X, 98% or more sequence identity with SB100X, 95% or more sequence identity with SB100X, 90% or more sequence identity with SB100X, 80% or more sequence identity with SB100X, or 70% or more sequence identity with SB100X.

In an embodiment, the complex (e.g., preferably a protein fusion) may comprise more than one degron tag. The one or more degron tags may be the same or may be different.

In an embodiment, the complex (e.g., preferably a protein fusion) may, in addition to a degron tag and a second protein further comprise a destabilizing domain. Destabilizing domains (DDs) are known in the art and described in a separate section of the disclosure of the present invention.

In another preferred embodiment, the ligand (i.e., signalling molecule) that reduces the enzymatic activity of the complex, e.g., preferably fusion protein, of the present invention comprising a degron tag and a second protein domain or protein is a Cereblon E3 Ligase Modulation Drug (CELMoDs). CELMoDs is a class of compounds known in the art and includes, for example, iberdomide, but is not limited thereto. In one embodiment, the ligand (i.e., signaling molecule) is iberdomide. In one embodiment, the ligand (i.e., signaling molecule) is a derivative from lenalidomide or pomalidomide. In one embodiment, the ligand (i.e., signaling molecule) is iberdomide CC-220 or CC-92480.

Physiological conditions as used herein refers primarily to the cellular environment to which a complex, e.g., preferably a protein fusion, is targeted when expressed in a cell or transfected or transduced as a protein into a cell. Depending on the specific properties of the complex, this may therefore be typically in an intracellular environment such as in the nucleus or in any other intracellular compartment that the protein is targeted to. In a preferred embodiment, the complex comprises a transposase and is translocated into the nucleus of a cell, to which in that case “physiological conditions” would refer to. A person skilled in the art can readily ascertain the physiological conditions that a given complex, e.g., preferably a fusion protein, is typically exposed to when expressed, transfected, or transduced.

As used herein, each occurrence of terms such as “comprising” or “comprises” may optionally be substituted with “consisting of” or “consists of”. Any embodiment of subject matter that is “comprising” or “comprises” a given feature is there expressly disclosed herein as the same embodiment that is “consisting” or “consists” of the same feature.

### Proteins to be Comprised in the Complexes of the Invention

In a preferred embodiment, the complex of the present invention comprises a destabilizing domain and a transposase domain.

In a preferred embodiment, the complex of the present invention comprises a degron tag and a transposase domain.

The complex of the invention is preferably a fusion protein.

Other alternative means to design and form a complex are known in art. As non-limiting examples, the complex may be in the form of or designed to be expressed as separate parts, e.g., using a binding domain that mediates transient interaction between the components of the complex after separate expression (such as, for example, SH3 domains, PDZ domains, GK domains, or GB domains), or using post-translation ligation such as, for example, via intein.

In a preferred embodiment, the complex is a fusion protein comprising a destabilizing domain and a transposase domain.

In another preferred embodiment, the complex is a fusion protein comprising a degron tag and a transposase domain.

In a preferred embodiment, the transposase domain comprised in the fusion protein of the transposase domain and the destabilizing domain of the present invention is the transposase domain of wild type Sleeping Beauty (SB100X). In an equally preferred embodiment, the destabilizing domain comprised in the fusion protein of the transposase domain and the destabilizing domain of the present invention is a destabilizing domain from E. coli-derived dihydrofolate reductase (ecDHFR).

In an even more preferred embodiment, the transposase domain comprised in the fusion protein of the transposase domain and the destabilizing domain of the present invention is the transposase domain of wild type Sleeping Beauty (SB100X) and the destabilizing domain comprised in the fusion protein of the transposase domain and the destabilizing domain of the present invention is a destabilizing domain from E. coli-derived dihydrofolate reductase (ecDHFR).

In a preferred embodiment, the transposase domain comprised in the fusion protein of the transposase domain and the degron tag of the present invention is the transposase domain of wild type Sleeping Beauty (SB100X). In an equally preferred embodiment, the degron tag comprised in the fusion protein of the transposase domain and the degron tag of the present invention is an IKZF3 zinc finger degron tag.

In an even more preferred embodiment, the transposase domain comprised in the fusion protein of the transposase domain and the degron tag of the present invention is the transposase domain of wild type Sleeping Beauty (SB100X) and the degron tag comprised in the fusion protein of the transposase domain and the degron tag of the present invention is an IKZF3 zinc finger degron tag.

The complex of the present invention, when in the form a fusion protein, may further comprise one or more linkers between the domains or components comprised in the fusion protein. A linker is a protein sequence that provides for flexibility in protein design by “linking” two otherwise unrelated proteins or protein domains together without affecting their tertiary structure. Suitable linkers depending upon application are known in the art.

### Nucleic Acid Vectors

Suitable nucleic acid-based vectors for expressing the complex, e.g., preferably fusion protein, of the invention in a cell of interest are known in the art, as well as methods for preparing a suitable vector.

The nucleic acid-based vector (nucleic acid vector) is not particularly limited as long as it is suitable for being introduced into the cell of interest expressing the complex, e.g., preferably fusion protein, of the invention. Non-limiting examples include plasmids, minicircle DNA, and mRNA.

Suitable features of DNA-based vectors for delivering and expressing the complex, e.g., preferably fusion protein, of the invention are known in the art.

Typically, a DNA-based vector will comprise a suitable promoter that will be transcribed in the cell of interest upon introduction of the vector. The promoter may be tailored to the specific application, e.g., it may be constitutive, heterologous, native, inducible, strong, weak, or otherwise optimized for the desired properties.

Other features of DNA-based vectors such as terminators, enhancers, regulatory sequences (e.g., upstream and/or downstream of the expressed sequence) may suitably be included.

mRNA vectors and suitable features for delivering the same into the cell of interest in order to deliver and express the complex, e.g., preferably fusion protein, of the invention to the cell of interest are known.

mRNA vectors may be optimized in the nucleotide makeup, for example, in their sequence, such as reducing the overall uridine content or modifying base compositions for optimizing translation. mRNA vectors may comprise modified bases, for example, pseudouridine (e.g., 5-methyl-pseudoruridine and/or N1-methyl-pseudouridine), which may improve the mRNA's properties such as translation, stability, and/or reduction of unwanted immunogenicity. Further suitable mRNA modifications are known in the art.

mRNA vectors may further comprise regulatory elements and/or modifications that improve the desired properties for delivering and expressing the complex, e.g., preferably fusion protein, of the invention to the cell of interest. For example, mRNA vectors may be modified in the 3′-UTR and/5′-UTR. As a non-limiting example, and mRNA vector may comprise microRNA binding sites, e.g., in one or both UTRs, that control expression by avoiding expression in undesired cell types that express a microRNA that can bind to the microRNA binding site included in the mRNA vector, while at the same time, the microRNA is not expressed in the desired cell type. Suitable microRNA binding sites, depending on the application, are known to a person skilled in the art.

Methods for producing mRNA are known in the art. For example, mRNA can be produced by in vitro transcription or by chemical synthesis.

Methods for introducing mRNA into a cell of interest are known in the art. For example, mRNA may be complexed with cationic polymers. Alternatively, or in addition, mRNA may be packaged into lipid particles such as lipid nanoparticles. Methods for preparing mRNA complexes amenable for introduction into a desired cell type are known in the art.

### Genetically Engineered Cells

The present invention provides methods for generating genetically engineered cells as well as genetically engineered cells obtained by the methods.

The methods of the present invention for generating genetically engineered cells generally involve contacting a cell of interest with the complex, e.g., preferably fusion protein, of the invention.

The cells of interest may be contacted with the complex directly, i.e., with the complex being in its mature protein form. Alternatively, the cells of interested may be contacted with a nucleic acid-based vector that encodes the complex, e.g., preferably fusion protein, of the invention respectively encodes the complex components. Suitable nucleic acid-based vectors are known in the art as well as described herein.

Typically, in addition to being contacted with the complex, e.g., preferably fusion protein, of the invention or a nucleic acid-based vector encoding the same, the cell of interest is further contacted with a donor that comprises genetic cargo to be delivered to the cell of interest to generate the desired resulting genetically engineered cell. In a preferred embodiment, the donor is a transposable element that comprises the genetic cargo to be delivered to the cell of interest, thereby generating the desired genetically engineered cell.

In a preferred embodiment, the donor is a transposable element from Sleeping Beauty, i.e., a transposable element that is amenable to mobilization and integration (i.e., transposition) by the Sleeping Beauty transposase (SB100X) and by complexes, e.g., preferably fusion proteins, of the invention comprising the Sleeping Beauty transposase (SB100X) domain.

Suitable donor and design thereof are known in the art. The design of a suitable donor to be used in the methods of the present invention will depend upon the transposase to be comprised in the complex, e.g., preferably fusion protein, of the invention that is to be used in the methods of the invention for generating genetically engineered cells. For example, in the case of the complex, e.g., preferably fusion protein, of the invention comprising a transposase domain from piggyBac, the skilled person is aware how to design and select a suitable donor that is amenable to mobilization and integration (i.e., transposition) by the piggyBac transposase.

In a preferred embodiment, the cell of interest to be modified by the methods of the present invention is an immune cell. In a preferred embodiment, the immune cell is obtained or to be obtained from a patient. In a preferred embodiment, the immune cell is obtained or to be obtained from a patient and modified ex vivo, resulting in a patient-specific genetically engineered immune cell. The patient-specific ex vivo genetically engineered immune cell is therefore generally compatible with the patient's immune system, i.e., compatible with the patient's MHC and HLAs.

In a preferred embodiment, the genetically engineered cells comprise, on average, 5 or fewer, 4 or fewer, 3 or fewer, or 2 or fewer copies of the genetic cargo (e.g., chimeric antigen receptor) integrated into their genome.

In a preferred embodiment, the genetically engineered cells comprise, on average, 5 or fewer, copies of the genetic cargo (e.g., chimeric antigen receptor) integrated into their genome.

In a preferred embodiment, the genetically engineered cells comprise, on average, 4 or fewer copies of the genetic cargo (e.g., chimeric antigen receptor) integrated into their genome.

In a preferred embodiment, the genetically engineered cells comprise, on average, 3 or fewer copies of the genetic cargo (e.g., chimeric antigen receptor) integrated into their genome.

In a preferred embodiment, the method of the present invention for generating genetically engineered cells, as described herein, the method is limited to a total maximum time period from contacting the cells of interest to obtaining the final genetically engineered cell product of 7 days, 6 days, 5 days, 4 days, 3 days, 2 days, or 1 day.

In a preferred embodiment, the method of the present invention for generating genetically engineered cells, as described herein, the method is limited to a total maximum time period from contacting the cells of interest to obtaining the final genetically engineered cell product of 7 days.

In a preferred embodiment, the method of the present invention for generating genetically engineered cells, as described herein, the method is limited to a total maximum time period from contacting the cells of interest to obtaining the final genetically engineered cell product of 6 days.

In a preferred embodiment, the method of the present invention for generating genetically engineered cells, as described herein, the method is limited to a total maximum time period from contacting the cells of interest to obtaining the final genetically engineered cell product of 5 days.

In a preferred embodiment, the method of the present invention for generating genetically engineered cells, as described herein, the method is limited to a total maximum time period from contacting the cells of interest to obtaining the final genetically engineered cell product of 4 days.

In a preferred embodiment, the method of the present invention for generating genetically engineered cells, as described herein, the method is limited to a total maximum time period from contacting the cells of interest to obtaining the final genetically engineered cell product of 3 days.

In a preferred embodiment, the method of the present invention for generating genetically engineered cells, as described herein, the method is limited to a total maximum time period from contacting the cells of interest to obtaining the final genetically engineered cell product of 2 days.

In a preferred embodiment, the method of the present invention for generating genetically engineered cells, as described herein, the method is limited to a total maximum time period from contacting the cells of interest to obtaining the final genetically engineered cell product of 1 day.

In a preferred embodiment, the population of genetically engineered cells obtained by the methods of the invention is substantially free of detectable protein levels of the complex by which the cells of interest were contacted 7 days after the initial contacting, 6 days after the initial contacting, 5 days after the initial contacting, 4 days after the initial contacting, 3 days after the initial contacting, 2 days after the initial contacting, 1 day after the initial contacting or on the same day of the initial contacting.

In a preferred embodiment, the population of genetically engineered cells obtained by the methods of the invention is substantially free of detectable protein levels of the complex by which the cells of interest were contacted 7 days after the initial contacting.

In a preferred embodiment, the population of genetically engineered cells obtained by the methods of the invention is substantially free of detectable protein levels of the complex by which the cells of interest were contacted 6 days after the initial contacting.

In a preferred embodiment, the population of genetically engineered cells obtained by the methods of the invention is substantially free of detectable protein levels of the complex by which the cells of interest were contacted 5 days after the initial contacting.

In a preferred embodiment, the population of genetically engineered cells obtained by the methods of the invention is substantially free of detectable protein levels of the complex by which the cells of interest were contacted 4 days after the initial contacting.

In a preferred embodiment, the population of genetically engineered cells obtained by the methods of the invention is substantially free of detectable protein levels of the complex by which the cells of interest were contacted 3 days after the initial contacting.

In a preferred embodiment, the population of genetically engineered cells obtained by the methods of the invention is substantially free of detectable protein levels of the complex by which the cells of interest were contacted 2 days after the initial contacting.

In a preferred embodiment, the population of genetically engineered cells obtained by the methods of the invention is substantially free of detectable protein levels of the complex by which the cells of interest were contacted 1 day after the initial contacting.

In a preferred embodiment, the population of genetically engineered cells obtained by the methods of the invention is substantially free of detectable protein levels of the complex by which the cells of interest were contacted one the same day of the initial contacting.

In a preferred embodiment, the cell of interest to be genetically engineered by the methods of the present invention is a T cell, e.g., a CD4 and/or CD8 positive cell.

In a preferred embodiment, the cell of interest to be genetically engineered by the methods of the present invention is an NK cell, NKT cell, gd T cell, macrophage, B cell, iPSC, iPSC-derived T cell, iPSC-derived NK cell, HSCs, or of any other somatic/mammalian cell type.

In a preferred embodiment, the genetic cargo to be delivered to the cell of interest to be genetically engineered with that cargo by the methods of the present invention is a cell surface receptor that is capable of binding to a desired cell surface antigen and thereby capable of targeting the resulting genetically engineered cell to patient cells expressing that cell surface antigen.

In a preferred embodiment, the genetic cargo to be delivered to the cell of interest to be genetically engineered with that cargo by the methods of the present invention is chimeric antigen receptor (CAR), T-cell receptor (TCR), a coreceptor, an immune fusion receptors, a sensor, or any gene of interest.

In a preferred embodiment, the cell surface receptor delivered as genetic cargo to the cells of interest obtained or obtainable from a patient is a chimeric antigen receptor (CAR) or a recombinant/engineered T cell receptor (TCR).

In a preferred embodiment, the cell surface receptor delivered as genetic cargo to the cells of interest obtained or obtainable from a patient is a chimeric antigen receptor (CAR).

In a preferred embodiment, the cell surface receptor delivered as genetic cargo to the cells of interest obtained or obtainable from a patient is a chimeric antigen receptor (CAR) and the cell of interest that is genetically engineered by the method of the invention is a T cell. In this embodiment, the resulting genetically engineered cell will generally be considered a “CAR-T cell” (i.e., a T cell modified with a chimeric antigen receptor).

In a preferred embodiment, the chimeric antigen receptor to be delivered to a patient's immune cell, e.g., preferably T cell, by the methods of the invention is capable of binding to a cell surface antigen that is predominantly or exclusively expressed by cancer cells. The resulting engineered immune cell will therefore be capable of targeting the patient's immune response to undesired cancer cells and hence be useful as a therapeutic in the treatment of cancer when administered to the patient.

This approach is generally known in the art as “adoptive immunotherapy”. It is advantageous in that it allows targeted growth inhibiting, preferably cytotoxic, treatment of tumor cells without the non-targeted toxicity to non-tumor cells that occurs with conventional treatments. In other words, it enables targeted treatment of cancer cells that express the selected cell surface antigen without the risk of affecting other cell types that do not express the antigen or express it only at low levels.

In a preferred embodiment, a chimeric antigen receptor to be delivered as genetic cargo by the methods of the present invention to cells of interest in order to obtain genetically engineered cells may comprises a costimulatory domain capable of mediating costimulation to immune cells. The costimulatory domain is preferably from 4-1BB, CD28, Ox40, ICOS or DAP10. The chimeric antigen receptor may further comprise a transmembrane domain, which is preferably a transmembrane domain from CD4, CD8 or CD28. The chimeric antigen receptor according the invention preferably further comprises a CAR spacer domain, e.g., from CD4, CD8, an Fc-receptor, an immunoglobulin, or an antibody. In a preferred embodiment, the spacer domain may be from or derived from IgG hinge regions such as from IgG3 hinge regions.

### Methods

The present invention provides methods for improving the properties of proteins used to generate genetically engineered cells.

The invention generally provides methods for modulating the half-life of complexes, e.g., preferably fusion proteins, comprising components that are useful for genetically engineering cells in a controlled and directed manner.

The invention also provides methods for modulating the enzymatic activity of complexes, e.g., preferably fusion proteins, comprising components that are useful for genetically engineering cells in a controlled and directed manner.

The methods of the invention for improving the properties of proteins can be used to generate genetically engineered cells. The methods generally involve providing and/or forming a complex of a destabilizing domain or a degron tag with a second protein domain or protein, e.g., a transposase domain.

The invention also provides methods for generating genetically engineered cells using the complexes, e.g., preferably fusion proteins, of the invention, as described herein. The invention encompasses the genetically engineered cells obtained or obtainable from the methods for generating genetically engineered cells, as described herein.

The inventive complexes and components of the complexes to be used in the methods of the invention are described herein.

In one embodiment, any specific method of the invention described and/or claimed herein can be performed exclusively in vitro, i.e., in one embodiment, any specific method is hereby expressly disclosed as an in vitro method.

In one embodiment, any specific method described and/or claimed herein is a method that is not a method for treatment of the human or animal body by surgery or therapy and diagnostic methods practised on the human or animal body.

Any product, i.e., complex, fusion protein, kit, substance, or composition disclosed and/r claimed herein is disclosed also for the specific use in the methods of the invention disclosed and claimed herein.

Any method step disclosed and/or claimed herein is expressly disclosed as being envisaged as having been performed outside of the scope of the method, i.e., as being recited in passive form in the method, without forming an active method step.

### Methods for Modulating the Half-Life of Complexes

The methods for modulating the half-life of complexes, e.g., preferably fusion proteins, of the invention, comprise linking a destabilizing domain or degron tag to a second protein domain or protein, thereby modulating the half-life of the complex compared to the second protein domain or protein without the destabilizing domain or degron tag.

The complex, e.g. preferably fusion protein, of the invention, comprising a destabilizing domain or degron tag that is capable of modulating the half-life of the complex is described herein.

In an embodiment, the method of the invention for modulating the half-life of a complex, e.g., preferably fusion protein, comprises linking a destabilizing domain, as described herein, to a second protein domain or protein, as described herein, e.g., a transposase domain. By linking the two components, the half-life of the complex can be modulated in a controlled and reversible manner.

Generally, linking a destabilizing domain to the second protein domain or protein, e.g., transposase domain, will cause a significant decrease in the half-life of the resulting complex, e.g., preferably fusion protein, of the destabilizing domain and the second protein domain or protein, e.g., transposase domain, as described herein.

The method of the invention for modulating the half-life of a complex, e.g., preferably fusion protein, comprising a destabilizing domain and a second protein domain or protein, e.g., a transposase domain, may further comprise contacting, after linking of the destabilizing domain to the second protein domain or protein, the resulting complex with a ligand (i.e., signalling molecule), thereby stabilizing the complex and restoring the half-life of the complex to a half-life greater than in the absence of the ligand.

In the method of the invention for modulating the half-life of a complex, as described above, contacting the complex, e.g., preferably fusion protein, with a ligand (i.e., signalling molecule) stabilizes complex under physiological conditions, resulting in the half-life of the complex being restored to at least about 10%, at least about 20%, at least about 30%, at least about 40%, at least about 50%, at least about 60%, at least about 70%, at least about 80%, at least about 90%, at least about 95%, at least about 98%, or at least about 99% of the half-life of the complex without being in contact with the ligand (i.e., signalling molecule) under the same physiological conditions.

In the method of the invention for modulating the half-life of a complex, as described above, linking the destabilizing domain to a second protein domain or protein, e.g., preferably as a fusion protein, destabilizes the resulting complex, resulting in the half-life of the complex being reduced at least about 10%, at least about 20%, at least about 30%, at least about 40%, at least about 50%, at least about 60%, at least about 70%, at least about 80%, at least about 90%, at least about 95%, at least about 98%, or at least about 99% under physiological conditions, compared to the second protein domain or protein without having been linked to the destabilizing domain under the same physiological conditions.

In a preferred embodiment, the method for modulating the half-life of a complex, e.g., preferably fusion protein, of the invention comprises linking the destabilizing domain from E. coli-derived dihydrofolate reductase (ecDHFR) to a second protein domain or protein. In an equally preferred embodiment, the method for modulating the half-life of a complex, e.g., preferably fusion protein, of the invention comprises linking a destabilizing domain to the transposase domain of wild type Sleeping Beauty (SB100X).

In a more preferred embodiment, the method for modulating the half-life of a complex, e.g., preferably fusion protein, of the invention comprises linking the destabilizing domain from E. coli-derived dihydrofolate reductase (ecDHFR) to the transposase domain of wild type Sleeping Beauty (SB100X).

In a preferred embodiment, the method for modulating the half-life of a complex, e.g., preferably fusion protein, of the invention comprises linking the destabilizing domain from E. coli-derived dihydrofolate reductase (ecDHFR) to the transposase domain of wild type Sleeping Beauty (SB100X), and further comprises contacting the complex with a ligand (i.e., signalling molecule). Contacting the complex fully or partially stabilizes the complex under physiological conditions, resulting in the half-life of the complex being restored to at least about 10%, at least about 20%, at least about 30%, at least about 40%, at least about 50%, at least about 60%, at least about 70%, at least about 80%, at least about 90%, at least about 95%, at least about 98%, or at least about 99% of the half-life of the complex without being in contact with the ligand (i.e., signalling molecule) under the same physiological conditions.

In a preferred embodiment, the ligand (i.e., signalling molecule) used to contact the complex in the method for modulating the half-life of the complex of the invention to fully or partially stabilize the complex and restore the half-life of the complex, as described above, is trimethoprim (TMP), shield-1, FK506/tacrolimus, or4-hydroxytamoxifen.

In one embodiment, in the method for modulating the half-life of a complex of the invention, the destabilizing domain is a destabilizing domain from a destabilizing domain from FK506 binding protein 12 (FKBP) and the ligand (i.e., signalling molecule) that, in the method, the complex is contacted by, to fully or partially stabilize the destabilizing domain, is shield-1 or FK506/tacrolimus.

In one embodiment, in the method for modulating the half-life of a complex of the invention, the destabilizing domain is a destabilizing domain human estrogen receptor ligand-binding domain and the ligand (i.e., signalling molecule) that, in the method, the complex is contacted by, to fully or partially stabilize the destabilizing domain, is 4-hydroxytamoxifen.

In preferred embodiment, in the method for modulating the half-life of a complex of the invention, the destabilizing domain is a destabilizing domain from E. coli-derived dihydrofolate reductase (ecDHFR) and the ligand (i.e., signalling molecule) that, in the method, the complex is contacted by, to fully or partially stabilize the destabilizing domain, is trimethoprim (TMP).

### Methods for Modulating the Enzymatic Activity of Complexes

The methods for modulating the enzymatic activity of complexes, e.g., preferably fusion proteins, of the invention, comprise linking a degron tag to a second protein domain or protein having enzymatic activity, e.g., transposase activity, thereby modulating the enzymatic activity of the complex that the second protein domain or protein confers, compared to the second protein domain or protein without degron tag, as described herein.

The method for modulating the enzymatic activity of complexes, e.g., preferably fusion proteins, of the invention, generally further comprises contacting the complex comprising the degron tag and the second protein domain or protein having enzymatic activity with a ligand (i.e., signalling molecule). Without being bound by theory, the interaction between the ligand and the complex comprising the degron tag and the second protein or protein domain having enzymatic activity may alter the activity of the complex that the second protein or protein domain confers.

In the method, binding of the ligand to the degron tag causes modulation of the enzymatic activity, e.g., transposases activity, that the complex exerts due to comprising the second protein domain or protein, which in itself exhibits the same activity, albeit possibly at other levels. Upon contacting the complex that the degron tag it is associated with, e.g., in the form a protein fusion, the enzymatic activity of the complex of the degron tag and the second protein under physiological conditions is reduced when compared to the activity of the same complex under the same physiological conditions in the absence of the ligand (i.e., signalling molecule).

In a preferred embodiment, in the method for modulating the enzymatic activity of a complex of the invention, contacting the complex with a ligand (i.e., signalling molecule) to primarily or exclusively modulates the activity of the complex that the degron tag is comprised in, and does not substantially trigger or prevent protein degradation, i.e., does not substantially affect the half-life of the complex under physiological conditions.

The complex, e.g. preferably fusion protein, of the invention, comprising a degron tag that is capable of modulating the enzymatic activity, e.g., transposase activity, of the complex is described herein.

In an embodiment, the method of the invention for modulating the enzymatic activity of a complex, e.g., preferably fusion protein, comprises linking a degron tag, as described herein, to a second protein domain or protein, as described herein, e.g., a transposase domain. By linking the two components, the enzymatic activity of the complex can be modulated in a controlled and reversible manner.

Generally, linking a degron tag to the second protein domain or protein, e.g., transposase domain, will in itself not result in a significant decrease in the enzymatic activity of the resulting complex, e.g., preferably fusion protein, that the second protein domain or protein, e.g., transposase domain, confers, as described herein.

In the method of the invention for modulating the enzymatic activity, e.g., transposase activity, of a complex, as described above, contacting the complex, e.g., preferably fusion protein, with a ligand (i.e., signalling molecule) reduces the activity of the complex under physiological conditions, resulting in the activity, e.g., transposases activity, of the complex being reduced by at least about 10%, at least about 20%, at least about 30%, at least about 40%, at least about 50%, at least about 60%, at least about 70%, at least about 80%, at least about 90%, at least about 95%, at least about 98%, or at least about 99% of the activity of the same complex without being in contact with the ligand (i.e., signalling molecule) under the same physiological conditions.

In a preferred embodiment of the method for modulating the enzymatic activity of a complex of the invention, the degron tag is an IKZF3 zinc finger degron tag.

In a preferred embodiment of the method for modulating the enzymatic activity of a complex of the invention, the ligand with which the complex is contacted in order to reduce the enzymatic activity of the complex, e.g., preferably fusion protein, of the present invention comprising a degron tag and a second protein domain or protein is an immunomodulatory imide drug (IMiD), as described herein. IMiDs is a class of compounds known in the art that generally encompasses thalidomide and its derivatives.

In a preferred embodiment of the method for modulating the enzymatic activity of a complex of the invention, the ligand with which the complex is contacted in order to reduce the enzymatic activity of the complex, e.g., preferably fusion protein, of the present invention comprising a degron tag and a second protein domain or protein is pomalidomide, lenalidomide, thalidomide, or a thalidomide analogue.

In a preferred embodiment of the method for modulating the enzymatic activity of a complex of the invention, the ligand with which the complex is contacted in order to reduce the enzymatic activity of the complex, e.g., preferably fusion protein, of the present invention comprising a degron tag and a second protein domain or protein is pomalidomide.

In a preferred embodiment of the method for modulating the enzymatic activity of a complex of the invention, the degron tag comprised in the complex, e.g., preferably fusion protein, of the invention comprising a degron tag and the second protein domain or protein to which the degron tag is linked in the method is an IKZF3 zinc finger degron tag, and the second protein domain or protein is a transposase domain.

In a more preferred embodiment of the method for modulating the enzymatic activity of a complex of the invention, the degron tag comprised in the complex, e.g., preferably fusion protein, of the invention comprising a degron tag and a second protein domain or protein to which the degron tag is linked in the method is an IKZF3 zinc finger degron tag, and the second protein domain or protein is a transposase domain from wild type Sleeping Beauty (SB100X).

In an even more preferred embodiment of the method for modulating the enzymatic activity of a complex of the invention, the degron tag comprised in the complex, e.g., preferably fusion protein, of the invention comprising a degron tag and a second protein domain or protein that the degron tag is linked to in the method is an IKZF3 zinc finger degron tag, and the second protein domain or protein is a transposase domain from wild type Sleeping Beauty (SB100X), and the enzymatic activity (i.e., transposition activity) of the complex is reduced upon contacting the complex, in the method, with a ligand (i.e., signaling molecule) under physiological conditions when compared to the same complex under the same physiological conditions in the absence of the ligand (i.e., without the contacting step of the method). In a preferred embodiment of the method for modulating the enzymatic activity of a complex of the invention, the ligand (i.e., signaling molecule) that mediates the reduction of enzymatic activity (i.e., reduced transposition activity) of the complex, e.g., preferably protein fusion, of the IKZF3 zinc finger degron tag and the transposase domain from wild type Sleeping Beauty (SB100X), that are linked to each other in the method, is an immunomodulatory imide drug (IMiD), thalidomide, or a thalidomide derivative. In an even more preferred embodiment of the method for modulating the enzymatic activity of a complex of the invention, in the method, contacting the complex with the ligand (i.e., signaling molecule) causes an reduction of enzymatic activity (i.e., reduced transposition activity) of the complex, e.g., preferably protein fusion, of the IKZF3 zinc finger degron tag and the transposase domain from wild type Sleeping Beauty (SB100X), and the ligand that the complex is contacted with is is pomalidomide or lenalidomide. In yet an even more preferred embodiment of the method for modulating the enzymatic activity of a complex of the invention, the ligand (i.e., signaling molecule) that the complex is contacted with, in the method, and that causes an reduction of enzymatic activity (i.e., reduced transposition activity) of the complex, e.g., preferably protein fusion, of the IKZF3 zinc finger degron tag and the transposase domain from wild type Sleeping Beauty (SB100X), is pomalidomide.

In a preferred embodiment of the method for modulating the enzymatic activity of a complex of the invention, the ligand (i.e., signaling molecule) by which the complex is contacted with causes a reduction of enzymatic activity (i.e., reduced transposition activity) of the complex, e.g., preferably protein fusion, of the IKZF3 zinc finger degron tag and the transposase domain from wild type Sleeping Beauty (SB100X), that have been linked according to the method, by at least about 10%, at least about 20%, at least about 30%, at least about 40%, at least about 50%, at least about 60%, at least about 70%, at least about 80%, at least about 90%, at least about 95%, at least about 98%, or at least about 99% under physiological conditions compared to the same complex under the same physiological conditions in the absence of the ligand, i.e., without having been contacted with the ligand. In a preferred embodiment, the ligand (i.e., signaling molecule) that the complex is contacted with in the method and that causes this reduction is pomalidomide.

In a preferred embodiment of the method for modulating the enzymatic activity of a complex of the invention, upon contacting the complex with pomalidomide according to the method, the residual enzymatic activity (i.e., transposition activity) of the complex, e.g., preferably fusion protein, of the IKZF3 zinc finger degron tag and the transposase domain from wild type Sleeping Beauty (SB100X) that have been linked according to the method of the invention is less than 10%, less than 9%, less than 8%, less than 7%, less than 6%, less than 5%, less than 4%, less than 3%, less than 2%, or less than 1% under physiological conditions of the activity of the same complex under the same physiological conditions in the absence of pomalidomide, i.e., without the complex having been contacted with the ligand.

In a preferred embodiment of the method for modulating the enzymatic activity of a complex of the invention, upon contacting the complex with pomalidomide according to the method of the invention, the residual enzymatic activity (i.e., transposition activity) of the complex, e.g., preferably fusion protein, of the IKZF3 zinc finger degron tag and the transposase domain from wild type Sleeping Beauty (SB100X) that have been linked in accordance with the method of the invention is less than 10% of the activity, under physiological conditions, of the same complex under the same physiological conditions in the absence of pomalidomide, i.e., without the complex having been contacted with the ligand.

In an even more preferred embodiment of the method for modulating the enzymatic activity of a complex of the invention, upon contacting the complex with pomalidomide according to the method of the invention, the residual enzymatic activity (i.e., transposition activity) of the complex, e.g., preferably fusion protein, of the IKZF3 zinc finger degron tag and the transposase domain from wild type Sleeping Beauty (SB100X) that have been linked in accordance with the method of the invention is less than 5% of the activity, under physiological conditions, of the same complex under the same physiological conditions in the absence of pomalidomide, i.e., without the complex having been contacted with the ligand.

In more preferred embodiment of the method for modulating the enzymatic activity of a complex of the invention, upon contacting the complex with pomalidomide according to the method of the invention, the residual enzymatic activity (i.e., transposition activity) of the complex, e.g., preferably fusion protein, of the IKZF3 zinc finger degron tag and the transposase domain from wild type Sleeping Beauty (SB100X) that have been linked in accordance with the method of the invention is less than 2% of the activity, under physiological conditions, of the same complex under the same physiological conditions in the absence of pomalidomide, i.e., without the complex having been contacted with the ligand.

In a preferred embodiment of the method for modulating the enzymatic activity of a complex of the invention, when contacting the complex with the ligand according to the method of the invention, the protein degradation rate and/or half-life of the complex, e.g., preferably fusion protein, of the IKZF3 zinc finger degron tag and the transposase domain from wild type Sleeping Beauty (SB100X) of the invention under physiological conditions is not substantially affected, i.e., is not affected by the presence of pomalidomide and effectively remains the same as without the complex having been contacted with the ligand.

In a preferred embodiment of the method for modulating the enzymatic activity of a complex of the invention, when contacting the complex with the ligand according to the method of the invention, the protein degradation rate and/or half-life of the complex, e.g., preferably fusion protein, of the IKZF3 zinc finger degron tag and the transposase domain from wild type Sleeping Beauty (SB100X) of the invention under physiological conditions when contacted by pomalidomide, according to the method of the invention, is the same as that of the same complex under the same conditions when no contacted by pomalidomide.

### Methods of Treatment and Medical Uses

The invention provides methods for improving the properties of proteins used to generate genetically engineered cells, which generally involve providing and/or forming a complex of a destabilizing domain or a degron tag with a second protein domain or protein, e.g., a transposase domain. The inventive complexes and components of the complexes as well as their use in methods for generating genetically engineered cells are described herein.

The present invention provides methods of using the improved complexes, e.g., preferably fusion proteins, of the invention, as described herein, to generate genetically engineered cells with enhanced safety, precision, and efficiency.

The complex of the invention, as described herein, that is provided or formed by the methods of the invention, i.e., a complex comprising a destabilizing domain or a degron tag and a second protein domain or protein, enables enhanced means for the generation of genetically engineered cells, such as ex vivo modification of patient T cells into CAR-T cells for the treatment of cancer. The methods and complexes of the invention provide improved precision, safety, and efficiency to generate genetically engineered cells such as patient-specific T cells for cancer treatment, as described herein.

The present invention provides a method for treatment, comprising a step of obtaining cells from a patient to thereby isolate the cells, contacting the isolated patient cells ex vivo with the complex, e.g., preferably fusion protein, of the invention, as described herein, and a donor, as described herein, to deliver genetic cargo to the patient cells, and administering the resulting genetically engineered cells to the patient, thereby treating the patient.

In a preferred embodiment, the method further comprises contacting the complex of the invention and the patient cells ex vivo with a ligand (i.e., signalling molecule), as described herein.

In a preferred embodiment, the complex used in the method of treatment is a complex, e.g., preferably fusion protein, of a destabilizing domain and a transposase domain, as described herein.

In an even more preferred embodiment, the complex used in the method of treatment is a complex, e.g., preferably fusion protein, comprising the destabilizing domain from E. coli-derived dihydrofolate reductase (ecDHFR) and the transposase domain of wild type Sleeping Beauty (SB100X), as described herein. In this embodiment, preferably, the method involves contacting the complex and the patient cells ex vivo with the ligand TMP.

In a preferred embodiment, the complex used in the method of treatment is a complex, e.g., preferably fusion protein, of a degron tag and a transposase domain, as described herein.

In an even more preferred embodiment, the complex used in the method of treatment is a complex, e.g., preferably fusion protein, comprises an IKZF3 zinc finger degron tag and the transposase domain of wild type Sleeping Beauty (SB100X), as described herein. In this embodiment, preferably, the method involves contacting the complex and the patient cells ex vivo with a ligand that is an an immunomodulatory imide drug (IMiD), as described herein. In a preferred embodiment, the ligand is pomalidomide, lenalidomide, thalidomide, or a thalidomide analogue. In a more preferred embodiment, the ligand is pomalidomide.

In a preferred embodiment, in the method of treatment of the present invention, once isolated and contacted with the complex of the invention, e.g., preferably fusion protein, and donor carrying the genetic cargo, the resulting population of genetically engineered patient cells is substantially free from detectable protein levels of the complex after 7 days, 6 days, 5 days, 4 days, 3 days, 2 days, 1 day or on the same day. In a preferred embodiment, the population is substantially free from detectable protein levels of the complex after 7 days.

In a preferred embodiment, the population is substantially free from detectable protein levels of the complex after 6 days. In a preferred embodiment, the population is substantially free from detectable protein levels of the complex after 5 days. In a preferred embodiment, the population is substantially free from detectable protein levels of the complex after 4 days. In a preferred embodiment, the population is substantially free from detectable protein levels of the complex after 3 days. In a preferred embodiment, the population is substantially free from detectable protein levels of the complex after 2 days. In a preferred embodiment, the population is substantially free from detectable protein levels of the complex after 1 day. In a preferred embodiment, the population is substantially free from detectable protein levels of the complex on the same day.

In this embodiment, the resulting genetically engineered patient cell population that is to be administered to the patient for treating the patient is exposed to the complex comprising a transposase for a significantly shorter duration than when contacted with an equal amount of the same transposase comprised in the complex or a similar vector encoding the same transposase. This improves safety, efficiency, reproducibility, and hence generally clinical effectiveness and quality of treatment.

In a preferred embodiment, in the method of treatment of the present invention, once isolated and contacted with the complex of the invention, e.g., preferably fusion protein, and donor carrying the genetic cargo, the resulting population of genetically engineered patient cells is exposed to significantly fewer transposition events (i.e., genomic integrations of the donor) compared to when contacted with an equal amount of the same transposase as comprised in the complex. This improves safety, efficiency, reproducibility, and hence generally clinical effectiveness and quality of treatment.

In a preferred embodiment, in the method of treatment of the invention, the isolated population of patient cells, after having been contacted by the complex of the invention and a donor carrying the genetic cargo to be delivered to the cells have, on average, 5 or fewer, 4 or fewer, 3 or fewer, or 2 or fewer copies of the genetic cargo integrated in their genome. In a preferred embodiment, the cells have 5 or fewer copies of the cargo integrated into their genome on average.

The present invention also provides a method for treatment by genetically modifying a cell of interest in vivo, comprising administering to a patient the complex, e.g., preferably fusion protein, of the invention, as described herein, to introduce a desired genetic cargo, as described herein, into cells of interest in the patient in vivo.

In one embodiment, the method for treatment by genetically modifying a cell of interest in vivo of the invention further comprises administering to the patient a donor carrying the genetic cargo, as described herein.

In one embodiment, the method for treatment by genetically modifying a cell of interest in vivo of the invention further comprises administering to the patient a ligand (i.e., signalling molecule) that modulates the half-life of the complex that is administered to the patient, as described herein.

In one embodiment, the method for treatment by genetically modifying a cell of interest in vivo of the invention further comprises administering to the patient a ligand (i.e., signalling molecule) that modulates the enzymatic of the complex that is administered to the patient, as described herein.

In one embodiment, the method for treatment by genetically modifying a cell of interest in vivo of the invention further comprises administering to the patient a ligand (i.e., signalling molecule) that modulates the half-life of the complex that is administered to the patient, as described herein and further comprises administering to the patient a ligand (i.e., signalling molecule) that modulates the enzymatic of the complex that is administered to the patient, as described herein.

In one embodiment of the method for treatment by genetically modifying a cell of interest in vivo of the invention, the complex, e.g., preferably fusion protein, of the invention is packaged into a nanoparticles. Suitable nanoparticles and methods for packaging nucleic acids and proteins are known in the art.

In one embodiment of the method for treatment by genetically modifying a cell of interest in vivo of the invention, the complex and/or donor are in the form of an AAV vector.

In one embodiment of the method for treatment by genetically modifying a cell of interest in vivo of the invention, the method further comprises administering to the patient a therapeutic AAV vector.

A treatment of cancer according to the present invention does not exclude that additional or secondary therapeutic benefits also occur in patients. For example, an additional or secondary benefit may be an enhancement of engraftment of transplanted hematopoietic stem cells that is carried out prior to, concurrently to, or after the treatment of cancer. However, it is understood that the primary treatment for which protection is sought is for treating the cancer itself, and any secondary or additional effects only reflect optional, additional advantages of the treatment of cancer growth.

The treatment of cancer according to the invention can be a first-line therapy, a second-line therapy, a third-line therapy, or a fourth-line therapy. The treatment can also be a therapy that is beyond is beyond fourth-line therapy. The meaning of these terms is known in the art and in accordance with the terminology that is commonly used by the US National Cancer Institute.

The present invention also provides the complexes, e.g., preferably fusion proteins, of the invention, as described herein, for use in any of the methods disclosed herein.

The present invention also provides pharmaceutical compositions comprising the complexes, e.g., preferably fusion proteins, of the invention, as described herein, for use in any of the methods disclosed herein.

In an embodiment, the use of the complexes or composition of the invention does not involve a step of obtaining cells from a patient and/or administering the cells to the patient.

### Kits

The present invention also provides a kit of the complex, e.g., preferably fusion protein, of the invention, as described herein, or a nucleic acid-based vector encoding the same, as described herein, and isolated patient cells as described herein (e.g., isolated immune cells such as T cells). In an embodiment, the kit further comprises a donor comprising a genetic cargo of interest, e.g., a transposon donor carrying a chimeric antigen receptor in the form of, e.g., a minicircle DNA or a plasmid.

### INDUSTRIAL APPLICABILITY

The complexes, compositions, and kits, as well as generally any embodiment of a product according to the present invention may be industrially manufactured and sold as products for the claimed methods and uses (e.g., for treating a cancer as defined herein), in accordance with known standards for the manufacture of pharmaceutical products.

Accordingly, the present invention is industrially applicable.

### EXAMPLES

### Materials and Methods

### 1. Plasmid Constructs: Generation of SB Transposase Fusion Variants

An expression vector comprising a T7 promotor and codon-optimized wild-type SB100X transposase was generated de novo in house using Gibson assembly. Synthetic minigenes comprising (i) a degron domain (Jan et al., 2021; Koduri et al., 2019) or the destabilization domains from FKBP12 (Banaszynski et al., 2006; Stankunas et al., 2003) or the destabilization domains from ecDHFR (Banaszynski et al., 2006; Iwamoto et al., 2010); (ii) a flexible linker KLGGGAPAVGGGPK (Kovac et al., 2020; Szuts and Bienz, 2000) and (iii) SB100X transposase were codon-optimized and generated de novo in house using Gibson assembly. The minigenes were cloned into the expression vector using specific restriction sites.

### 2. Tumor Cell Lines

The chronic myelogenous leukemia cell line K562 (ACC 10) was purchased from DSMZ (Deutsche Sammlung von Mikroorganismen und Zellkulturen, Braunschweig, Germany) and cultured in RPMI-1640 supplemented with 10% fetal calf serum (FCS), 2 mM glutamine and 100 U/mL penicillin/streptomycin. K562 expressing CD19 cell line was generated by transducing K562 cells with a lentiviral vector encoding a firefly luciferase (ffluc) and green fluorescent protein (GFP) transgene to enable detection by flow cytometry (GFP) and bioluminescence imaging (ffLuc) in mice, and bioluminescence-based cytotoxicity assays. HeLa cells were maintained in DMEM supplemented with 10% (vol/vol) FCS and 2 mM glutamine and 100 U/mL penicillin/streptomycin.

### 3. Transposition Assay

An transposon donor vector with neomycin resistance gene was generated de novo in house using Gibson assembly. In order to test the activity (in terms of integration capability) of the fusion-transposase (dd-SB100X) transposition assay was performed in HeLa cells as per the published protocol (Ivics et al., 1997). Briefly, ˜150,000 HeLa cells were seeded into each well of a 6-well plate a day before transfection. Cells were transfected with 500 ng of neo carrying transposon donor plasmid and 50 ng of transposase expression plasmid using jetOPTIMUS transfection reagent (Polyplus transfection, France) as per the manufacturer recommendations. 24 hours later, medium was replaced with fresh medium containing either DMSO or trimethoprim (TMP) and incubated for upto 3 days. The addition of TMP would stabilize the transposase (dd-SB100X) protein that can induce the transposition process. Samples from different conditions at different time points were harvested and stored at −20° C. for western blotting. For transposition assay 30,000 cells were seeded into 10 cm petri dish containing 10 mL of DMEM medium with G418 (400 μg/mL). Cells carrying the genomically integrated neomycin resistance gene due to SB-mediated transposition will survive neomycin selection (400 μg/mL). After 10 days of selection, cells were washed with PBS, fixed with tissue culture fixing solution, stained with methylene blue and counted.

### 4. Immunoblotting Analysis

Western blotting was performed for detecting the expression of the transposase protein (either wild type SB100X or the fusion-transposase, dd-SB100X). Briefly, cells were lysed in RIPA buffer containing protease inhibitor cocktail (Sigma-Aldrich) and cracked with liquid nitrogen for quick and efficient cell lysis. Cell lysates were centrifuged at 10,000×g for 10 min at 4° C. and the supernatants were collected and protein concentration was determined with DC Protein Assay Kit II (Bio Rad). 10 to 15 μg of protein were mixed with Laemmli sample buffer and boiled at 100° C. for 10 min and then loaded on a 4-20% precast gradient gel (Bio Rad). Proteins were transferred on methanol activated PVDF membrane using the Trans-Blot Turbo RTA Transfer Kit (Bio Rad). The membrane was blocked with blocking solution (5% milk in TBS-T) for 1 hour at room temperature and then incubated with primary anti-SB transposae antibody (R&D systems; 1:1000 dilution) and secondary anti-goat antibody (Sigma systems; 1:10000 dilution). Labelled protein were detected using clarity Western ECL substrate (Bio Rad) and ChemiDoc MP (Bio Rad). Membrane was stripped and was blocked again and incubated with primary anti-β-actin Ab (Sigma-Aldrich; 1:5000 dilution) followed by secondary anti-mouse antibodies (Bio Rad; 1:5000 dilution), to detect β-Actin loading control.

5. Generation of CAR T Cells from Healthy Human Blood Samples

Anonymous healthy donor blood samples were received upon written consent to participate in research protocols approved by the Institutional Review Board of the Universitätsklinikum Würzburg (UKW). Blood samples were processed for T cells isolation as published earlier (Querques et al., 2019). Briefly, Peripheral blood mononuclear cells (PBMCs) were isolated by density centrifugation with Biocoll separating solution (Biochrom). Both CD4+ and CD8+ T cells were sorted by negative isolation approach using microbeads and magnetic associated cell sorting (MACS, Miltenyi) methods as described by the manufacturer. The isolated CD4+ and CD8+ T cells were cultured in T cell medium (RPMI-1640 with 10% (vol/vol) human serum, 2 mM L-glutamine, 100 U ml-1 penicillin-streptomycin) containing IL-2 (50 units per mL) and activated by anti-CD3/CD28 bead stimulation (Thermo Fisher). Two days post-activation, approximately 2 million T cells were nucleofected with vectors (1 μg of CD19 CAR (EGFRt)-encoding transposon as plasmid or minicircle DNA; 1 μg of mRNA encoding SB transposase) or 10 μg of hsSB protein (Querques et al., 2019) using a 4D Nucleofector (Lonza) as per the manufacturer's instructions. CD19 CAR-modified (i.e., EGFRt+) T cells were enriched using biotin-conjugated anti-EGFR monoclonal antibody and anti-biotin beads (Miltenyi) as per the manufacturer's instructions. For assessing the functionality of engineered CAR T cells, various assays like cytotoxicity, proliferation and cytokine secretion were performed as per the standard protocols published earlier (Hudecek et al., 2013; Hudecek et al., 2010; Hudecek et al., 2015; Monjezi et al., 2017).

### 6. Transposon Copy Number Analysis in Human CAR T Cells

Genomic DNA (gDNA) of CAR-positive T cells was isolated using dPureLink gDNA Mini Kit (Invitrogen, California, USA). Droplet digital PCR (ddPCR) was set up and analyzed in technical triplicates. Each ddPCR reaction contained ready-to-use ddPCR Supermix for Probes No dUTP (Bio-Rad, California, USA), 20 ng of the respective fragmented gDNA, 0.2 μL of CviQl (10 000 U/mL) (NEB, Frankfurt am Main, Germany), 0.1 μL of Dpnl (20,000 U/mL) (NEB, Frankfurt am Main, Germany), 200 nM of each respective ddPCR FAM/HEX probe, and 600 nM of each respective forward and reverse primer in a final volume of 25 μL. CviQl was used to fragment the gDNA to ensure single-copies per droplet. DPNI was used to specifically digest non-integrated vectors. The ddPCR CARrm probe (FAM-agcagcatggtggcggcgct-BQH1), and primer CARrm forward (5′-atctggatgtcggggatcag-3′) and primer CARrm reverse (5′-gcttgctcaactctacgtct-3′) were designed to bind in the CAR sequence to amplify integrations resulting from CAR transposition events. Ribonuclease P/MRP 30 subunit (RPP30) gene was used as the copy number reference (two copies per genome) with ddPCR RPP30 probe (HEX-tggacctgcgagcgggttctgacc-BHQ1), RPP30forward (5′-ggttaactacagctcccagc-3′) and RPP30 reverse (5′-ctgtctccacaagtccgc-3′). QX200 droplet generator (Bio-Rad, California, USA) and PX1 PCR Plate Sealer (Bio-Rad, California, USA) were used to generate droplets and seal the reactions. PRISM and statistical analysis was performed using unpaired, two-tailed Student's T-test.

7. In Vitro Transcription of mRNA

Poly(A)-tailed ARCA-capped SB100X mRNA and dd-SB100 mRNA was produced by in vitro transcription from the expression plasmids (containing a T7-promotor, ref. to 1.) using the mMESSAGE mMACHINE kit and column purified using the MEGAclear kit (Ambion, Carlsbad, CA, USA).

### Results

### Development and Characterization of SB Transposase Fusion Variants

Sleeping Beauty transposase is a highly stable protein with a half-life of about 72-80 hours (Geurts et al., 2003; Mates et al., 2009). Developing novel strategies to shorten the half-life of SB transposase protein will further improve the safety profile of SB mediated gene delivery for clinical applications. Towards this goal we generated novel SB transposase fusion proteins that contain (i) an IKZF3 zinc finger degron tag, (ii) a FKBP12 destabilization domain, or (iii) an ecDHFR destabilization domain (FIG. 1A). These protein regulation systems are controlled by small molecules that either lead to rapid protein degradation (i) or stabilize the a priori unstable fusion protein (ii/iii) (FIG. 1B). We did not attempt to fuse the conditional destabilizing domains at the 3′ end (or the C-terminus) of the SB100X transposase as it was observed in the past that the C-terminal modifications lead to loss of transposition activity of SB transposase (Wilson et al., 2005; Yant et al., 2007).

The fusion transposase constructs were screened and validated in Lenti-X 293T cells for protein expression and regulation by small molecules (FIGS. 1C and 1D). Cells transfected with plasmid encoding ecDHFR domain containing fusion transposase (which we refer to as destabilized version of SB100X or dd-SB100X) could be stabilized in the presence of TMP whereas in the absence of TMP the fusion-protein was rapidly degraded (FIG. 1B, FIG. 1D). Immunoblot analysis clearly shows that the fusion-transposase protein could be stabilized in the presence of trimethoprim (at concentrations ranging from 10 nM to 1000 nM). In contrast, in the absence of trimethoprim, the protein was rapidly subjected to protein degradation (in DMSO control, FIG. 1D). Wild type SB transposase was used as a control for comparison.

Similarly, cells were transfected with plasmid that encoded a fusion transposase with a FKBP12 destabilizing domain (which we refer to as FKBP12-SB100X)—such that upon transcription and translation the fusion transposase protein is expected to be degraded in the absence of its ligand (shield-i or FK506) but in the presence of ligand the protein would be stabilized. Surprisingly, immunoblotting showed that there is no difference in protein levels regardless of the presence (at 100 nM or 10,000 nM) or absence of FK506, suggesting that this destabilizing domain was less suitable for use in conjunction with SB100X transposase (FIG. 1D).

Lastly, cells were transfected with plasmid that encoded a fusion transposase with IKZF3 zinc finger degron (which we refer to as degron-SB100X)—such that protein degradation is induced by pomalidomide and other thalidomide derivatives that recruit ubiquitin ligase complex for subsequent ubiquitination and protein degradation. In the absence of pomalidomide and other thalidomide analogues, the protein would be stable. Immunoblot analysis showed that indeed, there was less degron-SB100X fusion transposase detectable in the presence of pomalidomide (10 μM) compared to the DMSO control (FIG. 1D).

Based on these data, we focused on the degron-SB100X and dd-SB100X fusion transposases and characterized their activity and small-molecule-induced conditional protein regulation in human T cells.

### Characterization of Degron-SB100X Fusion Transposase

We first investigated if the IKZF3 zinc finger degron-fusion-transposase (degron-SB100X) protein retained transposition activity in human T cells. Activated T cells were nucleofected with a minicircle DNA transposon donor vector (expressing a CD19-CAR in cis with EGFRt as a reporter) and with either a plasmid expressing wild type SB transposase (SB100X) or fusion-transposase (dd-SB100X) at an optimal 1:1 molar ratio and cultured either in the presence of pomalidomide or DMSO as control. In T cells that had been nucleofected with the degron-SB100X fusion-transposase and treated with DMSO, we observed a similarly high gene transfer rate (^{˜}46.4%) as in T cells that had ben nucleofected with wild type SB transposase (SB100X) (^{˜}55.1%) (FIG. 1E). However, in T cells that had been nucleofected with the degron-SB100X fusion-transposase and treated with pomalidomide, the gene transfer rate was similarly low (^{˜}1.74%) as in T cells that had been nucleofected with minicircle DNA transposon donor vector alone (FIG. 1E).

Interestingly we observed that treatment with pomalidomide interfered with the activity of wild type SB100X (lacking the degron tag) and led to a reduction in gene transfer rate (^{˜}20.1%) compared to DMSO control (^{˜}52%). Taken together, the data show that the transposition activity of degron-SB100X fusion-transposase can be tightly controlled through the use of pomalidomide. The mechanism of action is reduced stability and shorter half-life of degron-SB100X fusion-transposase in the presence of pomalidomide, and interference of pomalidomide with the transposase.

The degron-tag fusion transposase technology can be used in various clinical applications to turn-on or turn-off transposition activity. Since pomalidomide or thalidomide derivatives are clinically available, the degron-SB100X fusion transposase can be readily implemented for controlling transposase activity in diverse cell and gene therapies, including in vivo applications.

Characterization of dd-SB100X Fusion Transposase

Next, we investigated the ecDHFR domain containing fusion transposase (dd-SB100X) system and validated in HeLa cells for its expression and activity either in the absence of in the presence of TMP (FIG. 2A, 2B). We hypothesized that treatment of cells with TMP would stabilize the fusion-transposase protein that can then be detected by immunoblotting whereas cells not treated with TMP would not stabilize the fusion-transposase protein (FIG. 2A).

The plasmid encoding the fusion-transposase (pdd-SB100X) along with the transposon was transfected into HeLa cells. For comparison, wild type SB100X plasmid along with the transposon was transfected as a positive control, and transposon alone was used as a negative control. Analysis of transposase expression at different time points (days 1, 2, and 3 after transfection) by immunoblotting showed that the wild type SB100X protein is expressed as 39 kDa protein (FIGS. 2B & 2C). In the presence of TMP, a ˜58 kDa fusion-transposase protein was detected, whereas in the absence of TMP, no residual fusion transposase protein was detectable after day 2 (FIG. 2C). These results demonstrate that the destabilization domain derived from ecDHFR triggers rapid degradation of the fusion-transposase (dd-SB100X) protein.

We next examined the activity of the fusion-transposase (dd-SB100X) by standard transposition assay wherein we transfected HeLa cells with a SB transposon donor plasmid encoding a neomycin resistance gene, together with plasmid encoding SB100X as a positive control, or the fusion-transposase protein. At 24 hours post-transfection, cells were treated with either TMP or DMSO (as a control) for 24 hours to 72 hours (FIG. 2B). From each time point, the same number of transfected cells (^{˜}30,000 cells) were seeded and subjected to antibiotic selection with G418. After 10 days, colonies were fixed, stained and counted to determine the integration events achieved under each of the experimental conditions. In the presence of TMP, the fusion-transposase protein was active and mediated transposition activity (FIG. 2D). In the absence of TMP, the fusion-transpose protein was degraded and transposition events were similar to background (transposon alone) (FIGS. 2C & 2D). Overall, these data show that the stability of ddSB100X fusion-transposase protein can be regulated through addition of TMP. Compared to wild type SB100X, the dd-SB100X fusion-transposase displayed reduced transposition activity in the presence of TMP. The reduced transposition activity of dd-SB100X could also be, in part, due to interference with the N-terminal DNA binding domain of the transposase. Even in the absence of the TMP, we observed a very low level of transposition activity, which might be due to the lag time required for the host cells to activate the degradation machinery.

Temporal Kinetic of dd-SB100X Fusion Transposase Expression in Human T Cells

Next, we evaluated the dd-SB100X fusion-transposase in human T cells. As evidenced by the immunoblot analysis, in the absence of TMP (Off state) the fusion-transposase protein (dd-SB100X) was rapidly subjected to degradation. While treatment with increasing concentrations of TMP (On state) led to an increase in fusion-transpose protein levels in a dose-dependent manner. The dd-SB100X fusion-transposase was completely stabilized when TMP was used at a concentration of (or above) 100 nM, and no further increase in protein level was observed at the 1000 nM and 2000 nM concentration. We elected to use TMP at 1000 nM for further experiments (FIG. 3A).

Next, we evaluated the temporal kinetic of dd-SB100X expression in the presence of TMP (1000 nM). As evidenced by the immunoblotting, the fusion-transposase (dd-SB100X) protein levels could be stabilized in a time dependent manner. The length of exposure to TMP is proportional to the amount of the stabilized-transposase protein within the cells (FIG. 3B). Culturing the T cells (CD4+& CD8+) in the presence of TMP for 24 hours resulted in a high amount of fusion-transposase protein in cells, whereas in the absence of TMP the amount of detectable protein was reduced (FIG. 3B and FIG. 3C). Interestingly, at 7 days post nucleofection, all the samples that had been nucleofected with dd-SB100X fusion-transposase plasmid were completely free of transposase protein, while the samples that had been nucleofected with wild type SB100X plasmid (pSB100X) and hsSB protein still had detectable transposase protein (FIG. 3B and FIG. 3C). Taken together, these data show that expression and activity of dd-SB100X fusion transposase can be controlled by TMP in a time and dose dependent manner.

Genomic and Functional Characterization of CAR T Cells Engineered with dd-SB100X Fusion-Transposase

We next investigated if the dd-SB100X fusion-transposase retained transposition activity in primary human T cells. We purified T cells from peripheral blood, activated T cells through CD3/CD28 stimulation and performed nucleofections of mincircle DNA transposon donor vector (encoding a CD19-CAR in cis with EGFRt as a reporter) and either a plasmid (p) expressing wild type SB transposase (SB100X) or fusion-transposase (dd-SB100X) at an optimal 1:1 ratio. In T cells that had been nucleofected with dd-SB100X and cultured in the presence of TMP after nucleofection, there was a high rate of gene transfer (^{˜}18%), even though lower compared to the wild type SB transposase (SB100X) (^{˜}53.4%) (FIG. 4A). In T cells that had been nucleofected with dd-SB100X and cultured in the absence of TMP after nucleofection, there was only a very low gene transfer rate (^{˜}6.6%) consistent with rapid degradation of dd-SB100X (FIG. 4A). We observed differences in CD19-CAR_EGFRt expression, as evidenced by mean fluorescence intensity (MFI) of EGFRt by flow cytometry. T cells that had been nucleofected with dd-SB100X fusion-transposase and cultured in the presence of TMP, had a lower MFI of EGFRt compared to T cells that were engineered using the wild type SB100X (FIG. 4A). Together, these data show that dd-SB100X fusion-transposase retains transposition activity in the presence of TMP in human T cells.

We next analyzed the relative number of transposon integrations on the genomic level and compared the functionality of CAR T cells that were engineered using either wild type transposase (SB100X) or the fusion-transposase (dd-SB100X). First, we enriched and expanded EGFRt+ T cells by anti-EGFRt antibody based magnetic bead selection on day 9 post-nucleofection (FIG. 4B). We then isolated genomic DNA (gDNA) from a fraction of the enriched CAR-positive cells and performed droplet digital PCR (ddPCR) for copy number analysis of the transposon carrying the CAR gene. We found that CAR T cells that had been engineered with dd-SB100X fusion transposase carried on average 2.99 copies per genome constituting significantly less integrations per genome in comparison to wild type SB-100X carrying 5.58 copies on average (FIG. 4C). Next, we evaluated and quantified the cytokine production (IL-2 and IFN-γ) after co-culturing with K562/CD19 target cells. We found that CAR T cells that had been engineered with dd-SB100X fusion-transposase produced similar levels of IL-2 and IFN-γ upon co-culture with K562/CD19 target cells as T cells that had been modified with wild type SB100X transposase (FIG. 4D). Moreover, CAR T cells that had been engineered with dd-SB100X fusion-transposase underwent productive proliferation (>3 cell divisions in 72 hours) upon stimulation with K562/CD19 target cells (FIG. 4E). Taken together, these data show that CAR T cells that are engineered with dd-SB100X fusion-transposase confer specific and potent anti-tumor functions and that this approach could provide a strategy to reduce transposon copy number in host cells, and subsequently the risk of genotoxicity.

Priming T Cells with TMP to Augment the Transposition Activity of dd-SB100X Fusion-Transposase

We sought to assess the effect of TMP priming on the transposition activity of dd-SB100 fusion-transposase. We primed CD8+ T cells with TMP (1000 nM) for 24 hours before nucleofection. Immediately after nucleofection, T cells were incubated either in the presence of TMP (1000 nM) or absence of TMP. T cells that had been primed with TMP and nucleofected with dd-SB100X fusion-transposase had a significant increase in gene transfer compared to T cells that had not been primed with TMP (^{˜}29.7% vs. ˜26.1%) (FIG. 5A). Moreover, interestingly we also observed differences in the mean fluorescence intensity (MFI) of EGFRt expression. CAR T cells that had been engineered with dd-SB100X fusion-transposase had a lower MFI compared to T cells that were engineered with wild type SB100X (FIG. 5A), consistent with a lower transposon copy number due to reduced activity of the dd-SB100X fusion transposase.

We enriched EGFRt+ T cells by anti-EGFRt antibody based magnetic bead selection on day 9 post-nucleofection (FIG. 5B). As another positive and desired effect of TMP priming, we found an increased yield of CAR T cell after 14 days of culture (FIG. 5B, bottom panel, left figure). On average, we obtained ˜19 million, ˜11 million, ˜13 million CD8+ CAR T cells with the wild type SB100X, the dd-SB100X fusion-transposase and the dd-SB100X fusion-transposase with TMP priming approach, respectively.

We next evaluated the cytolytic activity of the CAR T cells using K562/CD19 as target cells. CD19 CAR T cells showed potent cytolytic activity, eliminating ˜85% of the targets within 3 hours of co-culturing at an E:T ratio of 10:1 (FIGS. 5C & 5D). Overall, CAR T cells that had been engineered with fusion-transposase conferred similar potent and specific lysis as CAR T cells that had been engineered using wild type SB100X (FIGS. 5C & 5D).

Taken together, these data show that priming T cells with TMP prior to nucleofection with dd-SB100X fusion transposase, leads to increased gene transfer rates and increase yield of CAR T cells. CAR T cells that are engineered with dd-SB100X fusion-transposase confer specific and potent anti-tumor reactivity. TMP treatment had no adverse effects on the subsequent function of CAR T cells.

Evaluating the Transposition Activity of mRNA Encoding Fusion-Transposase (dd-SB100X)

We sought to encode dd-SB100X fusion-transposase as mRNA for nucleofection into T cells. We produced mRNA encoding either the wild type SB100X or the dd-SB100X fusion-transposase from their respective plasmids templates and performed gel electrophoresis that showed the expected band size and secondary structures (FIG. 6A).

Activated CD8+ T cells were primed with TMP for 24 hours before nucleofection. T cells were nucleofected with the minicircle DNA transposon donor vector (encoding CD19-CAR and EGFRt as a reporter) and mRNA encoding either wild type SB100X or dd-SB100X fusion-transposase at a ratio of 1:1. Immediately after nucleofection, T cells were incubated either in the absence or presence of TMP. T cells that were primed with TMP and nucleofected with mRNA encoding fusion-transposase (mRNA/dd-SB100X) exhibited a lower level of transposition activity (^{˜}3%) compared to T cells nucleofected with SB100X-mRNA (^{˜}45%) (FIG. 6B). CAR-expressing T cells were enriched using anti-EGFRt antibody based magnetic bead selection on day 9 post-nucleofection and expanded (FIG. 6C). Importantly, we observed lower MFI values for CAR T cells engineered with the dd-SB100X fusion-transposase mRNA compared to CAR T cells engineered with wild type SB100X mRNA (FIG. 6C).

We performed digital droplet PCR to determine the transposon copy number and found significantly lower values in CAR T cells that had been primed with TMP and nucleofected with mRNA encoding fusion-transposase (mRNA/dd-SB100X) compared to T cells that had been nucleofected with SB100X-mRNA (FIG. 6D). Collectively, these data demonstrate that the use of dd-SB100X leads to a desired lower transposon copy number in human CAR T cells compared to the use of wild type SB100X.

### Sequences

The nucleotide sequences encoding the amino acid sequences referred to in the present application are as follows:

>FKBP12-SB100X
SEQ ID NO: 1
ATGGGCGTGCAGGTCGAGACAATCTCTCCTGGCGACGGCAGAACATTCCCCAAGAGGGGCCA
GACATGCGTGGTGCACTATACCGGCATGCTGGAAGATGGCAAGAAGGTGGACAGCAGCCGGG
ACAGAAACAAGCCCTTCAAGTTCATGCTGGGCAAGCAAGAAGTGATCAGAGGCTGGGAAGAG
GGCGTCGCCCAGATGTCTGTTGGACAGAGAGCCAAGCTGACAATCAGCCCCGATTACGCCTA
TGGCGCCACAGGACACCCTGGCATCATTCCTCCACATGCCACACTGGTGTTCGACGTGGAAC
TGCTGAAGCCCGAG
AAACTTGGAGGCGGAGCACCAGCTGTTGGCGGCGGACCAAAG
ATGGGCAAGAGCAAAGAGATCAGCCAGGACCTGCGGAAGCGGATCGTGGATCTGCACAAGTC
TGGCTCTAGCCTGGGCGCCATCAGCAAGAGACTTGCGGTACCACGTTCATCTGTACAAACAA
TAGTACGCAAGTATAAACACCATGGGACCACGCAGCCGTCATACCGCTCAGGAAGGAGACGC
GTTCTGTCTCCTAGAGATGAACGTACTTTGGTGCGAAAAGTGCAAATCAATCCCAGAACAAC
AGCAAAGGACCTTGTGAAGATGCTGGAGGAAACAGGTACAAAAGTATCTATATCCACAGTAA
AACGAGTCCTATATCGACATAACCTGAAAGGCCACTCAGCAAGGAAGAAGCCACTGCTCCAA
AACCGACATAAGAAAGCCAGACTACGGTTTGCAACTGCACATGGGGACAAAGATCGTACTTT
TTGGAGAAATGTCCTCTGGTCTGATGAAACAAAAATAGAACTGTTTGGCCATAATGACCATC
GTTATGTTTGGAGGAAGAAGGGGGAGGCTTGCAAGCCGAAGAACACCATCCCAACCGTGAAG
CACGGGGGTGGCAGCATCATGTTGTGGGGGTGCTTTGCTGCAGGAGGGACTGGTGCACTTCA
CAAAATAGATGGCATCATGGACGCGGTGCAGTATGTGGATATATTGAAGCAACATCTCAAGA
CATCAGTCAGGAAGTTAAAGCTTGGTCGCAAATGGGTCTTCCAACACGACAATGACCCCAAG
CATACTTCCAAAGTTGTGGCAAAATGGCTTAAGGACAACAAAGTCAAGGTATTGGAGTGGCC
ATCACAAAGCCCTGACCTCAATCCTATAGAAAATTTGTGGGCAGAACTGAAAAAGCGTGTGC
GAGCAAGGAGGCCTACAAACCTGACTCAGTTACACCAGCTCTGTCAGGAGGAATGGGCCAAA
ATTCACCCAAATTATTGTGGGAAGCTTGTGGAAGGCTACCCGAAACGTTTGACCCAAGTTAA
ACAATTTAAAGGCAATGCTACCAAATACTAG
>ecDHFR-SB100X
SEQ ID NO: 2
ATGATCAGCCTGATCGCCGCTCTGGCCGTGGATTACGTGATCGGCATGGAAAACGCCATGCC
TTGGAACCTGCCTGCCGATCTGGCCTGGTTCAAGCGGAACACCCTGAACAAGCCCGTGATCA
TGGGCAGACACACCTGGGAGTCTATCGGCAGACCTCTGCCTGGCCGGAAGAACATCATCCTG
AGCAGCCAGCCTAGCACCGACGACAGAGTGACATGGGTCAAGAGCGTGGACGAAGCCATTGC
CGCTTGCGGAGATGTGCCTGAGATCATGGTTATCGGCGGAGGCAGAGTGATCGAGCAGTTCC
TGCCTAAGGCTCAGAAGCTGTACCTGACACACATCGACGCCGAGGTGGAAGGCGACACCCAC
TTTCCAGACTACGAGCCCGATGACTGGGAGAGCGTGTTCAGCGAGTTCCACGATGCCGACGC
TCAGAACAGCCACAGCTACTGCTTCGAGATCCTGGAAAGA
AAGCTCGGAGGCGGAGCCCCTGCTGTTGGCGGAGGACCAAAG
ATGGGCAAGAGCAAAGAGATCAGCCAGGACCTGCGGAAGCGGATCGTGGATCTGCACAAGTC
TGGCTCTAGCCTGGGCGCCATCAGCAAGAGACTTGCGGTACCACGTTCATCTGTACAAACAA
TAGTACGCAAGTATAAACACCATGGGACCACGCAGCCGTCATACCGCTCAGGAAGGAGACGC
GTTCTGTCTCCTAGAGATGAACGTACTTTGGTGCGAAAAGTGCAAATCAATCCCAGAACAAC
AGCAAAGGACCTTGTGAAGATGCTGGAGGAAACAGGTACAAAAGTATCTATATCCACAGTAA
AACGAGTCCTATATCGACATAACCTGAAAGGCCACTCAGCAAGGAAGAAGCCACTGCTCCAA
AACCGACATAAGAAAGCCAGACTACGGTTTGCAACTGCACATGGGGACAAAGATCGTACTTT
TTGGAGAAATGTCCTCTGGTCTGATGAAACAAAAATAGAACTGTTTGGCCATAATGACCATC
GTTATGTTTGGAGGAAGAAGGGGGAGGCTTGCAAGCCGAAGAACACCATCCCAACCGTGAAG
CACGGGGGTGGCAGCATCATGTTGTGGGGGTGCTTTGCTGCAGGAGGGACTGGTGCACTTCA
CAAAATAGATGGCATCATGGACGCGGTGCAGTATGTGGATATATTGAAGCAACATCTCAAGA
CATCAGTCAGGAAGTTAAAGCTTGGTCGCAAATGGGTCTTCCAACACGACAATGACCCCAAG
CATACTTCCAAAGTIGTGGCAAAATGGCTTAAGGACAACAAAGTCAAGGTATTGGAGTGGCC
ATCACAAAGCCCTGACCTCAATCCTATAGAAAATTTGTGGGCAGAACTGAAAAAGCGTGTGC
GAGCAAGGAGGCCTACAAACCTGACTCAGTTACACCAGCTCTGTCAGGAGGAATGGGCCAAA
ATTCACCCAAATTATTGTGGGAAGCTTGTGGAAGGCTACCCGAAACGTTTGACCCAAGTTAA
ACAATTTAAAGGCAATGCTACCAAATACTAG
>Degron-SB100X
SEQ ID NO: 3
ATGCACAAGAGGAGCCACACCGGCGAGAGGCCCTTCCAGTGCAACCAGTGCGGCGCCAGCTT
CACCCAGAAGGGCAACCTGCTGAGGCACATCAAGCTGCACACCGGCGAGAAGCCCTTCAAGT
GCCACCTGTGCAAC
AAGCTGGGCGGCGGCGCCCCCGCCGTGGGCGGCGGCCCCAAG
ATGGGAAAATCAAAAGAAATCAGCCAAGACCTCAGAAAAAGAATTGTAGACCTCCACAAGTC
TGGTTCATCCTTGGGAGCAATTTCCAAACGCCTGGCGGTACCACGTTCATCTGTACAAACAA
TAGTACGCAAGTATAAACACCATGGGACCACGCAGCCGTCATACCGCTCAGGAAGGAGACGC
GTTCTGTCTCCTAGAGATGAACGTACTTTGGTGCGAAAAGTGCAAATCAATCCCAGAACAAC
AGCAAAGGACCTTGTGAAGATGCTGGAGGAAACAGGTACAAAAGTATCTATATCCACAGTAA
AACGAGTCCTATATCGACATAACCTGAAAGGCCACTCAGCAAGGAAGAAGCCACTGCTCCAA
AACCGACATAAGAAAGCCAGACTACGGTTTGCAACTGCACATGGGGACAAAGATCGTACTTT
TTGGAGAAATGTCCTCTGGTCTGATGAAACAAAAATAGAACTGTTTGGCCATAATGACCATC
GTTATGTTTGGAGGAAGAAGGGGGAGGCTTGCAAGCCGAAGAACACCATCCCAACCGTGAAG
CACGGGGGTGGCAGCATCATGTTGTGGGGGTGCTTTGCTGCAGGAGGGACTGGTGCACTTCA
CAAAATAGATGGCATCATGGACGCGGTGCAGTATGTGGATATATTGAAGCAACATCTCAAGA
CATCAGTCAGGAAGTTAAAGCTTGGTCGCAAATGGGTCTTCCAACACGACAATGACCCCAAG
CATACTTCCAAAGTTGTGGCAAAATGGCTTAAGGACAACAAAGTCAAGGTATTGGAGTGGCC
ATCACAAAGCCCTGACCTCAATCCTATAGAAAATTTGTGGGCAGAACTGAAAAAGCGTGTGC
GAGCAAGGAGGCCTACAAACCTGACTCAGTTACACCAGCTCTGTCAGGAGGAATGGGCCAAA
ATTCACCCAAATTATTGTGGGAAGCTTGTGGAAGGCTACCCGAAACGTTTGACCCAAGTTAA
ACAATTTAAAGGCAATGCTACCAAATACTAG

### REFERENCES

Banaszynski, L. A., Chen, L. C., Maynard-Smith, L. A., Ooi, A. G., and Wandless, T. J. (2006). A rapid, reversible, and tunable method to regulate protein function in living cells using synthetic small molecules. Cell 126, 995-1004.
Bonger, K. M., Chen, L. C., Liu, C. W., and Wandless, T. J. (2011). Small-molecule displacement of a cryptic degron causes conditional protein degradation. Nat Chem Biol 7, 531-537.
Buchberger, A., Bukau, B., and Sommer, T. (2010). Protein quality control in the cytosol and the endoplasmic reticulum: brothers in arms. Mol Cell 40, 238-252.
Chan, T., Gallagher, J., Cheng, N.-L., Carvajal-Borda, F., Plummer, J., Govekung, A., Barrett, J. A., Khare, P. D., Cooper, L. J. N., and Shah, R. R. (2017). CD19-Specific Chimeric Antigen Receptor-Modified T Cells with Safety Switch Produced Under “Point-of-Care” Using the Sleeping Beauty System for the Very Rapid Manufacture and Treatment of B-Cell Malignancies. Blood 130, 1324-1324.
Chung, H. K., Jacobs, C. L., Huo, Y., Yang, J., Krumm, S. A., Plemper, R. K., Tsien, R. Y., and Lin, M. Z. (2015). Tunable and reversible drug control of protein production via a self-excising degron. Nat Chem Biol 11, 713-720.
Cocchiarella, F., Latella, M. C., Basile, V., Miselli, F., Galla, M., Imbriano, C., and Recchia, A. (2016). Transcriptionally regulated and nontoxic delivery of the hyperactive Sleeping Beauty Transposase. Mol Ther Methods Clin Dev 3, 16038.
de Macedo Abdo, L., Barros, L. R. C., Saldanha Viegas, M., Vieira Codeco Marques, L., de Sousa Ferreira, P., Chicaybam, L., and Bonamino, M. H. (2020). Development of CAR-T cell therapy for B-ALL using a point-of-care approach. Oncoimmunology 9, 1752592.
Galla, M., Schambach, A., Falk, C. S., Maetzig, T., Kuehle, J., Lange, K., Zychlinski, D., Heinz, N., Brugman, M. H., Gohring, G., et al. (2011). Avoiding cytotoxicity of transposases by dose-controlled mRNA delivery. Nucleic Acids Res 39, 7147-7160.
Geurts, A. M., Yang, Y., Clark, K. J., Liu, G., Cui, Z., Dupuy, A. J., Bell, J. B., Largaespada, D. A., and Hackett, P. B. (2003). Gene transfer into genomes of human cells by the sleeping beauty transposon system. Mol Ther 8, 108-117.
Gogol-Doring, A., Ammar, I., Gupta, S., Bunse, M., Miskey, C., Chen, W., Uckert, W., Schulz, T. F., Izsvak, Z., and Ivics, Z. (2016). Genome-wide Profiling Reveals Remarkable Parallels Between Insertion Site Selection Properties of the MLV Retrovirus and the piggyBac Transposon in Primary Human CD4(+) T Cells. Mol Ther 24, 592-606.
Hudecek, M., Izsvak, Z., Johnen, S., Renner, M., Thumann, G., and Ivics, Z. (2017). Going non-viral: the Sleeping Beauty transposon system breaks on through to the clinical side. Crit Rev Biochem Mol Biol 52, 355-380.
Hudecek, M., Lupo-Stanghellini, M. T., Kosasih, P. L., Sommermeyer, D., Jensen, M. C., Rader, C., and Riddell, S. R. (2013). Receptor affinity and extracellular domain modifications affect tumor recognition by ROR1-specific chimeric antigen receptor T cells. Clin Cancer Res 19, 3153-3164.
Hudecek, M., Schmitt, T. M., Baskar, S., Lupo-Stanghellini, M. T., Nishida, T., Yamamoto, T. N., Bleakley, M., Turtle, C. J., Chang, W. C., Greisman, H. A., et al. (2010). The B-cell tumor-associated antigen ROR1 can be targeted with T cells modified to express a ROR1-specific chimeric antigen receptor. Blood 116, 4532-4541.
Hudecek, M., Sommermeyer, D., Kosasih, P. L., Silva-Benedict, A., Liu, L., Rader, C., Jensen, M. C., and Riddell, S. R. (2015). The nonsignaling extracellular spacer domain of chimeric antigen receptors is decisive for in vivo antitumor activity. Cancer Immunol Res 3, 125-135.
Ivics, Z., Hackett, P. B., Plasterk, R. H., and Izsvak, Z. (1997). Molecular reconstruction of Sleeping Beauty, a Tc1-like transposon from fish, and its transposition in human cells. Cell 91, 501-510.
Ivics, Z., Katzer, A., Stuwe, E. E., Fiedler, D., Knespel, S., and Izsvak, Z. (2007). Targeted Sleeping Beauty transposition in human cells. Mol Ther 15, 1137-1144.
Iwamoto, M., Bjorklund, T., Lundberg, C., Kirik, D., and Wandless, T. J. (2010). A general chemical method to regulate protein stability in the mammalian central nervous system. Chem Biol 17, 981-988.
Jan, M., Scarfo, I., Larson, R. C., Walker, A., Schmidts, A., Guirguis, A. A., Gasser, J. A., Slabicki, M., Bouffard, A. A., Castano, A. P., et al. (2021). Reversible ON- and OFF-switch chimeric antigen receptors controlled by lenalidomide. Sci Transl Med 13.
Kebriaei, P., Singh, H., Huls, M. H., Figliola, M. J., Bassett, R., Olivares, S., Jena, B., Dawson, M. J., Kumaresan, P. R., Su, S., et al. (2016). Phase I trials using Sleeping Beauty to generate CD19-specific CAR T cells. J Clin Invest 126, 3363-3376.
Koduri, V., McBrayer, S. K., Liberzon, E., Wang, A. C., Briggs, K. J., Cho, H., and Kaelin, W. G., Jr. (2019). Peptidic degron for IMiD-induced degradation of heterologous proteins. Proc Natl Acad Sci USA 116, 2539-2544.
Kovac, A., Miskey, C., Menzel, M., Grueso, E., Gogol-Doring, A., and Ivics, Z. (2020). RNA-guided retargeting of Sleeping Beauty transposition in human cells. Elife 9.
Magnani, C. F., Tettamanti, S., Alberti, G., Pisani, I., Biondi, A., Serafini, M., and Gaipa, G. (2020). Transposon-Based CAR T Cells in Acute Leukemias: Where are We Going?Cells 9.
Maji, B., Moore, C. L., Zetsche, B., Volz, S. E., Zhang, F., Shoulders, M. D., and Choudhary, A. (2017). Multidimensional chemical control of CRISPR-Cas9. Nat Chem Biol 13, 9-11.
Mates, L., Chuah, M. K., Belay, E., Jerchow, B., Manoj, N., Acosta-Sanchez, A., Grzela, D. P., Schmitt, A., Becker, K., Matrai, J., et al. (2009). Molecular evolution of a novel hyperactive Sleeping Beauty transposase enables robust stable gene transfer in vertebrates. Nat Genet 41, 753-761.
Miskey, C., Amberger, M., Reiser, M., Prommersberger, S., Beckmann, J., Machwirth, M., Einsele, H., Hudecek, M., Bonig, H., and Ivics, Z. (2019). Genomic Analyses of SLAMF7 CAR-T Cells Manufactured by <em>Sleeping Beauty</em>Transposon Gene Transfer for Immunotherapy of Multiple Myeloma. bioRxiv, 675009.
Miyazaki, Y., Imoto, H., Chen, L. C., and Wandless, T. J. (2012). Destabilizing domains derived from the human estrogen receptor. Journal of the American Chemical Society 134, 3942-3945.
Monjezi, R., Miskey, C., Gogishvili, T., Schleef, M., Schmeer, M., Einsele, H., Ivics, Z., and Hudecek, M. (2017). Enhanced CAR T-cell engineering using non-viral Sleeping Beauty transposition from minicircle vectors. Leukemia 31, 186-194.
Nabet, B., Roberts, J. M., Buckley, D. L., Paulk, J., Dastjerdi, S., Yang, A., Leggett, A. L., Erb, M. A., Lawlor, M. A., Souza, A., et al. (2018). The dTAG system for immediate and target-specific protein degradation. Nat Chem Biol 14, 431-441.
Narayanavari, S. A., Chilkunda, S. S., Ivics, Z., and Izsvak, Z. (2017). Sleeping Beauty transposition: from biology to applications. Crit Rev Biochem Mol Biol 52, 18-44.
Natsume, T., and Kanemaki, M. T. (2017). Conditional Degrons for Controlling Protein Expression at the Protein Level. Annu Rev Genet 51, 83-102.
Nishimura, K., Fukagawa, T., Takisawa, H., Kakimoto, T., and Kanemaki, M. (2009). An auxin-based degron system for the rapid depletion of proteins in nonplant cells. Nat Methods 6, 917-922.
Peng, P. D., Cohen, C. J., Yang, S., Hsu, C., Jones, S., Zhao, Y., Zheng, Z., Rosenberg, S. A., and Morgan, R. A. (2009). Efficient nonviral Sleeping Beauty transposon-based TCR gene transfer to peripheral blood lymphocytes confers antigen-specific antitumor reactivity. Gene Ther 16, 1042-1049.
Prommersberger, S., Reiser, M., Beckmann, J., Danhof, S., Amberger, M., Quade-Lyssy, P., Einsele, H., Hudecek, M., Bonig, H., and Ivics, Z. (2021). CARAMBA: a first-in-human clinical trial with SLAMF7 CAR-T cells prepared by virus-free Sleeping Beauty gene transfer to treat multiple myeloma. Gene Ther.
Querques, I., Mades, A., Zuliani, C., Miskey, C., Alb, M., Grueso, E., Machwirth, M., Rausch, T., Einsele, H., Ivics, Z., et al. (2019). A highly soluble Sleeping Beauty transposase improves control of gene insertion. Nat Biotechnol 37, 1502-1512.
Raina, K., and Crews, C. M. (2010). Chemical inducers of targeted protein degradation. J Biol Chem 285, 11057-11060.
Ramanayake, S., Bilmon, I., Bishop, D., Dubosq, M. C., Blyth, E., Clancy, L., Gottlieb, D., and Micklethwaite, K. (2015). Low-cost generation of Good Manufacturing Practice-grade CD19-specific chimeric antigen receptor-expressing T cells using piggyBac gene transfer and patient-derived materials. Cytotherapy 17, 1251-1267.
Riordan, J. D., Drury, L. J., Smith, R. P., Brett, B. T., Rogers, L. M., Scheetz, T. E., and Dupuy, A. J. (2014). Sequencing methods and datasets to improve functional interpretation of sleeping beauty mutagenesis screens. BMC Genomics 15, 1150.
Singh, H., Huls, H., Kebriaei, P., and Cooper, L. J. (2014). A new approach to gene therapy using Sleeping Beauty to genetically modify clinical-grade T cells to target CD19. Immunol Rev 257, 181-190.
Stankunas, K., Bayle, J. H., Gestwicki, J. E., Lin, Y. M., Wandless, T. J., and Crabtree, G. R. (2003). Conditional protein alleles using knockin mice and a chemical inducer of dimerization. Mol Cell 12, 1615-1624.
Szuts, D., and Bienz, M. (2000). LexA chimeras reveal the function of Drosophila Fos as a context-dependent transcriptional activator. Proc Natl Acad Sci USA 97, 5351-5356.
Torikai, H., Moyes, J., and Cooper, L. (2017). Non-viral gene transfer with Sleeping Beauty system to engineer T cells for hematologic malignancies and solid tumors. Cell and Gene Therapy Insights 3, 301-311.
Voigt, K., Gogol-Doring, A., Miskey, C., Chen, W., Cathomen, T., Izsvak, Z., and Ivics, Z. (2012). Retargeting sleeping beauty transposon insertions by engineered zinc finger DNA-binding domains. Mol Ther 20, 1852-1862.
Weber, E. W., Parker, K. R., Sotillo, E., Lynn, R. C., Anbunathan, H., Lattin, J., Good, Z., Belk, J. A., Daniel, B., Klysz, D., et al. (2021). Transient rest restores functionality in exhausted CAR-T cells through epigenetic remodeling. Science 372.
Wilber, A., Frandsen, J. L., Geurts, J. L., Largaespada, D. A., Hackett, P. B., and Mclvor, R. S. (2006). RNA as a source of transposase for Sleeping Beauty-mediated gene insertion and expression in somatic cells and tissues. Mol Ther 13, 625-630.
Wilson, M. H., Kaminski, J. M., and George, A. L., Jr. (2005). Functional zinc finger/sleeping beauty transposase chimeras exhibit attenuated overproduction inhibition. FEBS Lett 579, 6205-6209.
Yant, S. R., Huang, Y., Akache, B., and Kay, M. A. (2007). Site-directed transposon integration in human cells. Nucleic Acids Res 35, e50.

1. A complex, comprising:
A) a destabilizing domain or degron tag; and
B) a second protein domain or protein having enzymatic activity,
wherein the destabilizing domain or degron tag is capable of modulating the half-life of the complex and/or is capable of modulating the enzymatic activity of the complex, and
wherein the complex is capable of binding to a signaling molecule or ligand.

2. The complex of claim 1, wherein the complex is a fusion protein comprising the destabilizing domain or degron tag and the second protein domain or protein.

3. The complex of any one of claims 1-2, wherein the second protein domain or protein is a transposase domain and the enzymatic activity is transposase activity.

4. The complex of any one of claims 1-3, wherein the destabilizing domain or degron tag is selected from:
a) a destabilizing domain from E. coli-derived dihydrofolate reductase (ecDHFR),
b) a destabilization domain from FK506 binding protein 12 (FKBP), or
d) a degron tag, optionally wherein the degron tag is an IKZF3 zinc finger degron tag.

5. The complex of any one of claims 1-4, wherein the destabilizing domain or degron tag is linked to the second protein domain or protein at the N-terminus.

6. The complex of any one of claims 1-5, wherein the complex is a fusion protein and further comprises a linker, wherein the destabilizing domain or degron tag is linked to the second protein domain or protein via a linker.

7. The complex of any one of claims 1-6, wherein the destabilizing domain or degron tag modulates the half-life of the complex when exposed to one or more signaling molecule(s) or ligand(s), optionally wherein the destabilizing domain or degron tag reduces the half-life of the complex compared to the half-life of the second protein domain or protein when not in complex with the destabilizing domain or degron tag.

8. The complex of any one of claims 1-7, wherein the destabilizing domain or degron tag modulates the half-life of the complex when exposed to one or more signaling molecule(s) or ligand(s), optionally wherein the half-life of the complex is increased when the complex is exposed to the one or more signaling molecule(s) or ligand(s) compared to the same complex in absence of the one or more signaling molecule(s) or ligand(s).

9. The complex of any one of claims 1-8, wherein the destabilizing domain or degron tag modulates the enzymatic activity of the complex.

10. The complex of any one of claims 1-9, wherein the destabilizing domain or degron tag modulates the enzymatic activity of the complex when exposed to one or more signaling molecule(s) or ligand(s), optionally wherein the enzymatic activity of the complex is reduced when the complex is exposed to the one or more signaling molecule(s) or ligand(s) compared to the same complex in absence of the one or more signaling molecule(s) or ligand(s).

11. The complex of any one of claims 1-10, wherein the one or more signaling molecule(s) or ligand(s) is/are selected from:
a) immunomodulatory imide drugs,
b) pomalidomide, lenalidomide, or thalidomide derivatives
b) trimtheoprim,
c) shield-1 or FK506/tacrolimus.

12. The complex of any one of claims 1-11, wherein the one or more signaling molecule(s) or ligand(s) comprises or is pomalidomide.

13. The complex of any one of claims 1-12, wherein the one or more signaling molecule(s) or ligand(s) comprises or is trimtheoprim.

14. The complex of any one of claims 1-13, wherein the one or more signaling molecule(s) or ligand(s) reduces the enzymatic activity of the complex by at least about 10%, at least about 20%, at least about 30%, at least about 40%, at least about 50%, at least about 60%, at least about 70%, at least about 80%, at least about 90%, at least about 95%, at least about 98%, or at least about 99% compared to the same complex under the same physiological conditions in the absence of the ligand.

15. The complex of any one of claims 1-14, wherein in the presence of the one or more signaling molecule(s) or ligand(s), the residual enzymatic activity of the complex is less than 10%, less than 9%, less than 8%, less than 7%, less than 6%, less than 5%, less than 4%, less than 3%, less than 2%, or less than 1% of the activity of the same complex in the absence of the one or more signaling molecule(s) or ligand(s).

16. The complex of any one of claims 1-15, wherein the complex is destabilized in the absence of the one or more signaling molecule(s) or ligand(s), such that the half-life of the complex is reduced by at least about 10%, at least about 20%, at least about 30%, at least about 40%, at least about 50%, at least about 60%, at least about 70%, at least about 80%, at least about 90%, at least about 95%, at least about 98%, or at least about 99% when compared to the same complex in the presence of the ligand.

17. The complex of any one of claims 1-16, wherein contacting the complex with the one or more signaling molecule(s) or ligand(s) stabilizes the complex, such that the half-life of the complex is restored to at least about 10%, at least about 20%, at least about 30%, at least about 40%, at least about 50%, at least about 60%, at least about 70%, at least about 80%, at least about 90%, at least about 95%, at least about 98%, or at least about 99% of the half-life of the same complex when not in contact with the ligand.

18. The complex of any one of claims 1-17, wherein the second protein domain or protein is a transposase domain is or is derived from Sleeping Beauty, PiggyBac, Tol2, Frog Prince, TcBuster, Mos1, or Hellraiser.

19. The complex of any one of claims 1-18, wherein the second protein domain or protein is a transposase domain from Sleeping Beauty or a transposase domain from Sleeping Beauty, optionally wherein the transposase domain is SB100X of wild type Sleeping Beauty or is derived from SB100X.

20. The complex of any one of claims 1-19, wherein:
a) the transposase domain is SB100X of wild-type Sleeping Beauty, the destabilizing domain or degron tag is a destabilizing domain from E. coli-derived dihydrofolate reductase (ecDHFR), optionally wherein the one or more signaling molecule(s) or ligand(s) comprises or is trimtheoprim (TMP); or
b) the transposase domain is SB100X of wild-type Sleeping Beauty, the destabilizing domain or degron tag is is an IKZF3 zinc finger degron tag, optionally wherein the one or more signaling molecule(s) or ligand(s) comprises or is pomalidomide.

21. The complex of any one of claims 1-20, wherein the complex is a fusion protein and comprises an amino acid sequence having at least 70%, at least 80%, at least 85%, at least 90%, at least 95%, at least 98%, or at least 99% sequence identity to an amino acid sequence encoded by the nucleotide sequence of any one of SEQ ID NOs: 1-3.

22. The complex of any one of claims 1-20, wherein the complex is a fusion protein and wherein the fusion protein comprises or consists of the amino acid sequence encoded by the nucleotide sequence of any one of SEQ ID NOs: 1-3.

23. A nucleic acid, encoding the complex or components of the complex as defined in any one of claims 1-22.

24. The nucleic acid of claim 23, wherein the nucleic acid is an mRNA.

25. A composition or pharmaceutical composition, comprising the complex of nucleic acid encoding as defined in any one of claims 1-24.

26. A method of modulating the half-life of a complex,
wherein the complex is as defined in any one of claims 1-22,
the method comprising linking the second protein domain or protein to a destabilizing domain or a degron tag,
wherein the destabilizing domain or degron tag is capable of modulating the half-life of the complex,
wherein the destabilizing domain or degron tag is as defined in any one of claims 1-22,
wherein the second protein or protein domain is as defined in any one of claims 1-22.

27. The method of claim 26, further comprising contacting the complex with one or more signaling molecule(s) or ligand(s) as defined in any one of claims 1-22.

28. A method of modulating the enzymatic activity of a complex,
wherein the complex is as defined in any one of claims 1-22,
the method comprising linking the second protein domain or protein to a destabilizing domain or a degron tag,
wherein the destabilizing domain or degron tag is capable of modulating the enzymatic of the complex,
wherein the destabilizing domain or degron tag is as defined in any one of claims 1-22,
wherein the second protein or protein domain is as defined in any one of claims 1-22.

29. The method of claim 28, further comprising contacting the complex with one or more signaling molecule(s) or ligand(s) as defined in any one of claims 1-22.

30. A method for producing a genetically engineered cell,
the method comprising contacting a cell of interest with a complex as defined in any one of claims 1-22 or with a nucleic acid encoding the fusion protein as defined in any one of claims 23-24.

31. The method of claim 30, further comprising contact the cell with a donor.

32. The method of claim 31, wherein the donor is a transposon donor that is a transposable element comprising a genetic cargo to be delivered to the cell.

33. The method of claim 32, wherein the transposon donor is a plasmid or a minicircle DNA.

34. The method of any one of claims 30-33, wherein the genetic cargo comprises a chimeric antigen receptor (CAR).

35. The method of any one of claims 30-34, wherein the method is limited to a total maximum time period from contacting the cells of interest to obtaining the final genetically engineered cell product of 7 days, 6 days, 5 days, 4 days, 3 days, 2 days, or 1 day.

36. A genetically engineered cell obtained by the method as defined in any one of claims 30-35.

37. The genetically engineered cell as defined in claim 36 for use in a method of treating a disease.

38. The genetically engineered cells for use of claim 37, wherein the cell is an immune cell and wherein the disease is cancer.

39. The genetically engineered cell for use of claim 38, wherein the immune cell is a T cell and the genetic cargo delivered to the T cell is a chimeric antigen receptor (CAR) targeting a surface antigen expressed by the cancer.

40. The genetically engineered cell of any one of claims 36-39, wherein the cell comprises 10, 9, 8, 7, 6, 5 or fewer than 5 copies of the genetic cargo integrated into its genome.

41. The genetically engineered cell of claim 40, wherein the cell comprises no more than 5 copies of the genetic cargo integrated into its genome.

42. A method for treatment, comprising a step of obtaining cells from a patient to thereby isolate the cells, contacting the isolated patient cells ex vivo with the complex, as defined in any one of claims 1-22 and a donor, as defined in any one of claims 31-34, to deliver genetic cargo to the patient cells, and administering the resulting genetically engineered cells to the patient, thereby treating the patient.

43. The method of claim 42, wherein the method further comprises contacting the complex and the patient cells ex vivo with one or more signaling molecule(s) or ligand(s) as defined in any one of claims 1-22.
